(19) United States
(12) Patent Application Publication
(10) Pub. No.: US 2024/0076741 A1
(43) Pub. Date: Mar. 7, 2024

(54) METHOD FOR PREIMPLANTATION GENETIC SCREENING OF EMBRYOS FOR DETECTION OF STRUCTURAL REARRANGEMENTS

(71) Applicant: REPROBIOGEN INC., Toronto (CA)

(72) Inventors: Svetlana MADJUNKOVA, Toronto (CA); Clifford L. LIBRACH, Toronto (CA)

(21) Appl. No.: 18/506,654

(22) Filed: Nov. 10, 2023

Related U.S. Application Data
(63) Continuation-in-part of application No. 17/083,803, filed on Oct. 29, 2020, now Pat. No. 11,814,682.

(51) Int. Cl. C12Q 1/6883; C12Q 1/686

(52) U.S. Cl. CPC C12Q 1/6883; C12Q 1/686; C12Q 2600/156

(57) ABSTRACT

A method is described for determining carrier status of an embryo for a chromosomal rearrangement prior to implantation of the embryo. The method involves obtaining cells from the embryo from at least day 4 post in vitro fertilization; conducting long-read nanopore sequencing of the DNA of the cells and the chromosomal rearrangement carrier parent to detect at least one breakpoint; preparing primers specific to the breakpoint; employing the primers in a polymerase chain reaction customized to the breakpoint (cBP-PCR) to determine whether the breakpoint is indicative of chromosomal rearrangement; and determining on the basis of cBP-PCR whether the embryo status is chromosomal rearrangement carrier or chromosomal rearrangement noncarrier; and conducting Sanger sequencing to determine balance in the embryo. The method can assist chromosomal rearrangement carrier parents to pursue reproductive technologies while preventing vertical transmission of a chromosomal rearrangement to offspring.

### CROSS REFERENCE TO RELATED APPLICATIONS

This application is a continuation in part of, and claims the benefit of U.S. patent application Ser. No. 17/083,803 filed Oct. 29, 2020, entitled METHOD FOR PREIMPLANTATION GENETIC SCREENING OF EMBRYOS FOR DETECTION OF STRUCTURAL REARRANGEMENTS.

### FIELD

The present disclosure relates generally to a method for preimplantation testing of embryos to determine and reduce transmission of balanced chromosomal rearrangements (BCRs).

### BACKGROUND

Carriers of balanced chromosomal rearrangements (BCRs) are at risk for infertility, recurrent miscarriages, and abnormal offspring because of unbalanced rearrangements or translocation. A small subgroup of carriers is at risk for neurodevelopmental or neuropsychiatric conditions (Halgren et al., 2018).

Preimplantation genetic testing for structural rearrangements (PGT-SR) allows for the preselection of embryos without chromosomal gains or losses (Munne et al., 2000). However, next-generation, low-pass, whole-genome sequencing, which is the current standard for preimplantation aneuploidy (PGT-A) screening of cells from an embryo biopsy, cannot distinguish between an embryo that carries a BCR and one that does not. Thus, such technology cannot provide information to prospective parents to enable prevention of vertical transmission of the BCR to future generations, nor can such technology detect cryptic potential disease-causing imbalances, complex rearrangements, or gene disruptions at breakpoint sites.

Indirect approaches to genetic-linkage analysis used in PGT-SR, such as single-nucleotide polymorphism (SNP) array or sequencing-based haplotyping, are labor intensive, costly, and limited to familial cases of BCRs because DNA from family members who are known BCR carriers or from embryos with chromosomal unbalances resulting from BCR is required to enable haplotype phasing (Vermeesch et al., 2016). In addition, such approaches cannot detect potential disease-causing cryptic micro-deletions or micro-duplications.

Another technique, mate-pair next-generation sequencing which is also labor intensive, can delineate chromosomal structural rearrangements with abnormal phenotypes but may not perform well when the breakpoint is in a highly repetitive genomic region (Aristidou et al., 2017).

A preimplantation genetic test to discriminate embryos that carry a BCR from those that do not, as well as for detecting cryptic imbalances and/or complex rearrangements, is desirable. Embryos so assessed will have an increased likelihood of success in reproductive technologies.

### SUMMARY

It has been found that that a method that permits determination of an embryo's carrier status for a balanced chromosomal rearrangement (BCR) prior to implantation can help prevent vertical transmission of a BCR to the offspring of a BCR carrier parent.

There is described herein a method of determining carrier status of an embryo for a chromosomal rearrangement prior to implantation of the embryo. The method comprises obtaining cells of the embryo from a trophectoderm biopsy at least day 4 post in vitro fertilization; conducting long-read nanopore sequencing and data analysis of the DNA of the cells and the DNA of the parent carrier of chromosomal rearrangement to detect at least one breakpoint, wherein the sequencing is conducted on DNA fragments prepared from the DNA of the parent carrier of chromosomal rearrangement and from the DNA of the cells, wherein said DNA fragments comprise ultra long fragments of 50 kb or greater, and said DNA fragments comprise an average fragment length of from about 5 to 20 kb, wherein the chromosomal rearrangement of the parent carrier comprises a translocation, reciprocal translocation, deletion, duplication, pericentric inversion, or paracentric inversion; preparing customized primers specific to the breakpoint; employing the customized primers in a polymerase chain reaction customized to the breakpoint (cBP-PCR) to determine whether the breakpoint is indicative of chromosomal rearrangement; determining on the basis of cBP-PCR whether the embryo status is chromosomal rearrangement carrier or chromosomal rearrangement noncarrier; and determining, on the basis of Sanger sequencing, whether the chromosomal rearrangement carrier embryo is fully balanced.

The cells from trophectoderm biopsy may comprise 3 to 10 trophectoderm cells, and may be obtained from the embryo at day 5 or 6 post in vitro fertilization.

As to the step of conducting long-read nanopore sequencing, the DNA fragments may be prepared and sequenced having an average length of from about 8 to 15 kb (for example, 14 kb), and/or the DNA fragments are prepared and sequenced comprising ultra long fragments of 100 kb or greater. The long-read nanopore sequencing may be conducted using a real-time long-read nucleic acid sequencer for up to 48 hours. A chromosomal copy-number variation (CNV) plot may be generated from the long-read nanopore sequencing. For detection of multiple breakpoints, customized primers may be prepared for each breakpoint. The Sanger sequencing may be conducted at such a high sensitivity so as to determine full balance at single base resolution. Further, the breakpoint may be detected in a highly repetitive genomic region.

The method may comprise implantation of the embryo into a human subject if chromosomal rearrangement noncarrier status is indicated. Further, an option step of freezing the embryo may be utilized if chromosomal rearrangement noncarrier status is indicated, as well as the step of thawing the frozen embryo and implanting said embryo into a human subject.

The chromosomal rearrangement carrier status of the embryo to be determined may be a balanced chromosomal rearrangement, a cryptic imbalance or a complex rearrangement. Further, the chromosomal rearrangement may comprise a balanced chromosomal rearrangement selected from the group consisting of inversion, translocation and Robertsonian translocation. Unbalanced rearrangements to be determined may be, for example, an insertion or a deletion. Chromosomal rearrangements occurring de novo can be detected.

There is described herein a method of determining carrier status of an embryo for a chromosomal rearrangement prior to implantation of the embryo, said method comprising: obtaining cells of the embryo from a trophectoderm biopsy at least day 4 post in vitro fertilization; conducting long-read nanopore sequencing and data analysis of the DNA of the cells and the DNA of the parent carrier of the chromosomal rearrangement to detect at least one breakpoint; preparing customized primers specific to the breakpoint; employing the customized primers in a polymerase chain reaction customized to the breakpoint (cBP-PCR) to determine whether the breakpoint is indicative of chromosomal rearrangement; determining on the basis of cBP-PCR whether the embryo status is chromosomal rearrangement carrier or chromosomal rearrangement noncarrier; and determining, on the basis of Sanger sequencing, whether the chromosomal rearrangement carrier embryo is fully balanced.

The cells from trophectoderm biopsy may comprise 3 to 10 trophectoderm cells. The chromosomal rearrangement of the at least one carrier parent may comprise a reciprocal translocation, a pericentric inversion, or paracentric inversion. In the step of conducting long-read nanopore sequencing, the DNA fragments may be prepared and sequenced having an average length of from about 5 to 20 kb, from about 8 to 15 kb, for example 14 kb, or from about 8 to 10 kb. In the step of conducting long-read nanopore sequencing, the DNA fragments that are prepared and sequenced may comprise ultra long fragments of 50 kb or greater, or 100 kb or greater. Such ultra long fragments may be included even when the DNA fragments have an average length of 50 to 20 kb, for example 8 to 15 kb.

There is described herein a method for determining carrier status of an embryo for a balanced chromosomal rearrangement (BCR) prior to implantation of the embryo, said method comprising: obtaining cells/trophectoderm biopsy (3-10 trophectoderm cells) from the embryo from at least day 4 post in vitro fertilization; conducting long-read nanopore sequencing of the DNA of the parent, carrier of BCR, and of the cells from the embryo; and conducting long-read sequencing data analysis to detect at least one breakpoint; preparing customized primers specific to the breakpoint; employing the customized primers in a polymerase chain reaction customized to the breakpoint (cBP-PCR) to determine whether the breakpoint is indicative of BCR; determining on the basis of cBP-PCR whether the embryo status is BCR carrier or BCR noncarrier; and determining, on the basis of Sanger sequencing, whether the BCR carrier embryo is fully balanced down to single base resolution.

The method may be used for conducting pre-implantation genetic screening of embryos for chromosomal rearrangements that are balanced or unbalanced. Insertions, deletions, duplications, inversions, translocations, reciprocal translocations, Robertsonian translocations may be detected. Chromosomal rearrangements that occur de novo can be detected.

Other aspects and features of the present disclosure will become apparent to those ordinarily skilled in the art upon review of the following description of specific embodiments in conjunction with the accompanying figures.

### BRIEF DESCRIPTION OF THE FIGURES

Embodiments of the present disclosure will now be described, by way of example only, with reference to the Figures.

FIG. 1 depicts a schematic methodology for preimplantation genetic testing for structural rearrangements (PGT-SR) with a comprehensive test employing the use of long-read nanopore sequencing with breakpoint confirmation to differentiate embryos for use in reproductive technologies.

FIG. 2 shows that comprehensive methodology depicted in FIG. 1 can differentiate euploid embryos as carrier versus noncarrier.

FIG. 3 depicts a schematic of breakpoint detection using a nanopore MINION™ sequencer. Breakpoints in the parent, TC-1 and affected child were found on both chromosomes. Breakpoint locations were consistent with the karyotyping coordinates but were ˜500 kb and ˜2 Mb upstream from the breakpoints identified for chr 8 and for chr 22 with SNP array, respectively.

FIG. 4 depicts a schematic of custom primer design, based on breakpoints and corresponding normal chromosomes, for amplification was via PCR (cBP-PCR).

FIG. 5 depicts screen shots of the analysis conducted for chr 8 (upper) and chr 22 (lower).

FIG. 6 depicts a chromatographic separation for validation based on the balanced carrier (TC-1); affected child (TC-1_P); non-carrier DNA (TC-1_1-father); and on low template WGA based on a sample of 5 single lymphocytes from the mother (TC-1_SC), thus validating a correct determination of carrier status.

FIG. 7 depicts a CNV plot generated using whole genome sequencing data from a MINION™ sequencer compared to standard short read sequencing for a euploid, XX embryo (EMB-2, TC-1).

FIG. 8 depicts a CNV plot generated using whole genome sequencing data from a MINION™ sequencer compared to standard short read sequencing for an aneuploid, XY embryo with trisomy 16 and 21 and the del/dup associated with unbalanced translocation del8q24.3, dup22q13.31 (EMB-6, TC-1).

FIG. 9 depicts the result of a custom designed breakpoint PCR using established MINION™ sequencer breakpoint mapping strategy. Breakpoint amplification is present in the parent (TC-10-1) and embryos balanced translocation carriers (EMB-8 and 10), and absent in the parent (TC-10), embryo EMB-2 and child born after transferring this embryo (TC-10-2).

FIG. 10 depicts SNP haplotyping using Karyomapping Infinium Arrays visualized using BlueFuse multi software. Arrow indicates the region of interest that was karyotype guided. EMB-2 inherited normal paternal allele (P2) corresponding to the haplotype and karyotype of the child born after transfer of this embryo.

FIG. 11 depicts results from custom breakpoint (cBP) PCR in couple TC-3 undergoing PGT-SR. Comprehensive PGT-SR on the embryos was performed using the assay based on MINION™ sequencer breakpoint mapping of parental samples.

FIG. 12 depicts results from custom breakpoint (cBP) PCR in couple TC-8 undergoing PGT-SR. Comprehensive PGT-SR on the embryos was performed using the assay based on MINION™ sequencer breakpoint mapping of parental samples.

FIG. 13 depicts results from custom breakpoint (cBP) PCR in couple TC-11 undergoing PGT-SR. Comprehensive PGT-SR on the embryos was performed using the assay based on MINION™ sequencer breakpoint mapping of parental samples. Preimplantation and postnatal results from cBP-PCR on child born after embryo transfer is in full concordance.

FIG. 14 depicts schematic results from TC-11 (see FIG. 13) of Sanger sequencing of the breakpoints of TC-11, revealing an insertion of 22 bp at junction site, that was transmitted to both embryos balanced translocation carriers (EMB-3 and EMB-15).

FIG. 15 pertains to translocation of chr 8 and chr 22, and provides a schematic of a diagnostic strategy for comprehensive PGT-SR utilizing nanopore sequencing.

FIG. 16 shows custom breakpoint amplification for use in the diagnostic strategy for comprehensive PGT-SR utilizing nanopore sequencing. Standard NGS-based PGT-SR diagnosed 3 of the embryos to be balanced/euploid (EMB 2: 46,XX, 3: 46,XX and 5: 46,XX) and one aneuploid (EMB 6: 48,XY,+16,+21). cBP PCR determined that 1 of the 3 euploid embryos was a balanced carrier (EMB 2 46,XX, t(8,22)(q24.3,q13.31)), and the other 2 (EMB 3 and 5, 46,XX) were non-carriers.

FIG. 17 depicts a schematic overview of the testing of TC-1 embryos in the identification of one of the 3 euploid embryos as a balanced carrier.

FIG. 18 illustrates a workflow scheme for breakpoint detection in BCR carriers using a parent karyotype (genetic report) guided approach, as described in Example 2.

### DETAILED DESCRIPTION

The method described herein permits reproductive technologies that prevent vertical transmission of a balanced chromosomal rearrangement (BCR) to the offspring of a BCR carrier.

A method of determining carrier status of an embryo for a chromosomal rearrangement is described herein, which may be used prior to implantation of the embryo. The method comprising obtaining cells of the embryo from a trophectoderm biopsy at least day 4 post in vitro fertilization. A long-read nanopore sequencing and data analysis of the DNA of the cells is then conducted, and the DNA of the parent carrier of a chromosomal rearrangement may also be conducted so as to detect at least one breakpoint.

Sequencing is conducted on DNA fragments prepared from the DNA of the parent carrier of chromosomal rearrangement and from the DNA of the cells. Chromosomal rearrangement de novo can also be detected in the DNA of the cells of the embryo using the method. The DNA fragments may comprise ultra long fragments of 50 kb or greater, and may comprise an average fragment length of from about 5 to 20 kb. The chromosomal rearrangement of the parent carrier may comprise a translocation, reciprocal translocation, Robertsonian translocation, deletion, insertion, duplication, pericentric inversion, or paracentric inversion. Customized primers are prepared that are specific to the breakpoint, and the customized primers are utilized in a polymerase chain reaction customized to the breakpoint (cBP-PCR) to determine whether the breakpoint is indicative of chromosomal rearrangement.

Subsequently, it is determined on the basis of cBP-PCR whether the embryo status is indicated as being a carrier of the chromosomal rearrangement or is a noncarrier; and if a carrier, a determination can be made on the basis of Sanger sequencing, whether the chromosomal rearrangement carrier embryo is fully balanced.

The cells from trophectoderm biopsy may comprise 3 to 10 trophectoderm cells, which may be obtained from the embryo at day 5 or 6 post in vitro fertilization, for example.

For conducting long-read nanopore sequencing, the DNA fragments may be prepared and sequenced having an average length of from about 8 to 15 kb, for example 14 kb, or may be within in a range such as 8-10 kb. In the step of conducting long-read nanopore sequencing, the DNA fragments may be prepared and sequenced comprising ultra long fragments of 100 kb or greater. The long-read nanopore sequencing may be conducted using a real-time long-read nucleic acid sequencer for up to 48 hours. A chromosomal copy-number variation (CNV) plot may be generated from the long-read nanopore sequencing. Multiple breakpoints may be detected, and customized primers may be prepared for each breakpoint. Sanger sequencing may be conducted to determine full balance at single base resolution. Advantageously, the method may be used when the breakpoint is detected in a highly repetitive genomic region.

The method may encompass implantation of the embryo into a human subject if chromosomal rearrangement noncarrier status is indicated. The optional step of freezing the embryo may be conducted, if chromosomal rearrangement noncarrier status is indicated, in which case thawing of the frozen embryo and implanting the embryo into a human subject are also encompassed. The chromosomal rearrangement carrier status of the embryo to be determined may be a balanced chromosomal rearrangement, a cryptic imbalance or a complex rearrangement. The chromosomal rearrangement may comprise a balanced chromosomal rearrangement, such as inversion or translocation.

Further, there is provided herein a method of determining carrier status of an embryo for a balanced chromosomal rearrangement (BCR) prior to implantation of the embryo, the method comprising: obtaining cells/trophectoderm biopsy (3-10 trophectoderm cells) from the embryo from at least day 4 post in vitro fertilization; conducting long-read nanopore sequencing of the DNA of the parent, carrier of BCR, and of the embryo cells, and conducting long-read sequencing data analysis to detect at least one breakpoint; preparing customized primers specific to the breakpoint; employing the customized primers in a polymerase chain reaction customized to the breakpoint (cBP-PCR) to determine whether the breakpoint is indicative of BCR; determining on the basis of cBP-PCR whether the embryo status is BCR carrier or BCR noncarrier; and determining on the basis of Sanger sequencing whether a BCR carrier embryo is fully balanced down to single base resolution.

The method may advantageously be employed when at least one parent of the embryo is a BCR carrier. For example, such a carrier parent may possess a translocation, a reciprocal translocation, a Robertsonian translocation, a pericentric inversion, or a paracentric inversion.

The cells of the embryo to be assessed may be obtained from the embryo later than day 4 post in vitro fertilization, for example at day 5 or 6 post in vitro fertilization.

The average length of sequenced DNA fragments in the long-read nanopore sequencing may be from about 5 to 20 kb, for example from about 8 to 15 kb, for example from about 8 to 10 kb. Lengthy or “ultra-long” sequences may be included in the sequencing, for example greater than 50 kb, or greater than 100 kb. Ultra-long sequences may be included even when the average fragment length is smaller, such as from about 5 to 20 kb, 8 to 15 kb (for example 14 kb), or 8 to 10 kb.

The long-read nanopore sequencing is conducted using a MINION™ sequencer or a GRIDION™ high throughput sequencer (Oxford Nanopore Technologies, Oxford UK). A typical period of time for long-read nanopore sequencing may be, for example, 48 hours.

In the described method, a chromosomal copy-number variation (CNV) plot may be generated from the long-read nanopore sequencing.

In the described method, multiple breakpoints can be detected, and customized primers can be prepared for one, multiple or all breakpoints. Sanger sequencing is conducted to confirm the at least one breakpoint, and full balance can be detected at single base resolution.

Once the embryo is determined to have BCR noncarrier status, the embryo may be implanted into a human subject. Optionally, the embryo may be frozen if BCR noncarrier status is indicated, so as to be ready for later use. Once frozen, such an embryo can be properly stored and later thawed for implanting into a human subject at the desired time.

The preimplantation testing described herein, permits the parents to determine which embryo to select in order to avoid vertical transmission of a BCR to offspring. Further, the method would allow de novo variant/rearrangement detection.

Advantageously, the method describes permits accurate discrimination between embryos that carry a balanced chromosomal rearrangement (BCR) and embryos that do not. Advantageously, the determination can be made even when the breakpoint site is in a highly repetitive genomic region. The presence of cryptic imbalances and/or of complex rearrangements may be determined using the described method.

The described method can determine carrier status of an embryo for a chromosomal rearrangement prior to implantation of the embryo, that may occur de novo. Such a method comprises obtaining cells of the embryo from a trophectoderm biopsy at least day 4 post in vitro fertilization; conducting long-read nanopore sequencing and data analysis of the DNA of the cells to detect at least one breakpoint; preparing customized primers specific to the breakpoint; employing the customized primers in a polymerase chain reaction customized to the breakpoint (cBP-PCR) to determine whether the breakpoint is indicative of chromosomal rearrangement; determining on the basis of cBP-PCR whether the embryo status is carrier or non-carrier for the chromosomal rearrangement to be detected; and determining, on the basis of Sanger sequencing, whether the chromosomal rearrangement carrier embryo comprises the subject rearrangement prior to implantation.

To gain a better understanding of the invention described herein, the following example is set forth, based on multiple embryos from multiple couples pursuing a pregnancy through a reproductive technology. It should be understood that these examples are for illustrative purposes only. Therefore, they should not limit the scope of this invention in anyway.

### Example 1

Comprehensive Test for Preimplantation Detection of Translocations in Human Embryos

Summary

A brief summary of this Example is presented below, followed by detailed background information, methods and results.

This Example involves the direct sequencing of long and ultra-long DNA strands (between tens and hundreds of thousands of kilobases) in real time, with minimal capital cost and user time, on a small hand-held platform (Cretu Stancu et al., 2017). Using this technology, the results of a karyotype-guided PGT-SR protocol was clinically validated by means of low-coverage, long-read sequencing to identify breakpoints, which were manually reviewed and then validated by means of polymerase chain reaction and Sanger sequencing.

Briefly, eleven couples, with one partner who was a BCR carrier, underwent in vitro fertilization at the CReATe Fertility Centre in Toronto between 2016 and 2018. Of the 11 couples, 9 underwent in vitro fertilization with the use of PGT-SR because of a known BCR (seven reciprocal translocations, one pericentric inversion, and one paracentric inversion), and the other 2 couples involved patients in whom a balanced translocation was incidentally discovered after aberrations indicative of a BCR were detected on routine PGT-A analysis. A MINION™ sequencer was used to identify the precise breakpoints in all of the 11 BCR carriers.

Biopsy of the trophectoderm (four to six cells) was performed on 82 blastocysts from these 11 couples, followed by whole-genome amplification with the use of the SUREPLEX™ DNA Amplification System (Illumina) or the REPLI-G™ amplification kit (Qiagen) on the trophectoderm DNA. Standard PGT-SR was performed on all the cell samples from the embryos by means of short-read next-generation sequencing-based testing with the VERISEQ™ PGS (preimplantation genetic screening) kit and the Karyomapping SNP array (Illumina). Of the 82 blastocysts, 27 (33%) were euploid and the rest 55 (67%) were aneuploid or unbalanced.

Using a comprehensive approach of PGT-SR with a MINION™ sequencer, it was found that 11 of the 27 euploid embryos (41%) carried a BCR; the remainder (16 [59%]) tested negative. The comprehensive approach was thus able to distinguish BCR carriers from noncarriers and map the breakpoints with single-base resolution in the embryos that were carriers. These results were confirmed through haplotype phasing and with the genotypes of the six children born to date from this cohort. Concurrent aneuploidy detection is also possible with the use of the MINION™ sequencer, and the results from eight representative cases are presented herein below.

The results presented in this example show that a clinical application of long-read nanopore sequencing for preimplantation testing can distinguish embryos that carry a BCR from those that do not and thus can serve in the prevention of vertical transmission of BCRs to the offspring of BCR carriers.

Background

Carriers of balanced chromosomal rearrangements (BCRs) are at risk for infertility, recurrent miscarriages, and abnormal offspring because of unbalanced rearrangements. A small subgroup of carriers is at risk for neurodevelopmental or neuropsychiatric conditions (Halgren et al., 2018). Preimplantation genetic testing for structural rearrangements (PGT-SR) allows for the preselection of embryos without chromosomal gains or losses (Munne et al., 2000). However, next-generation, low-pass, whole-genome sequencing, which is the current standard for preimplantation aneuploidy (PGT-A) screening of cells from an embryo biopsy, cannot distinguish between an embryo that carries a BCR and one that does not and thus does not provide information to the prospective parents that would enable the prevention of vertical transmission of the BCR to future generations and the detection of cryptic potential disease-causing imbalances, complex rearrangements, or gene disruptions at breakpoint sites. Indirect approaches to genetic-linkage analysis used in PGT-SR, such as single-nucleotide polymorphism (SNP) array or sequencing-based haplotyping, are labor intensive, costly, and limited to familial cases of BCRs because DNA from family members who are known BCR carriers or from embryos with chromosomal unbalances resulting from BCR is required to enable haplotype phasing (Vermeesch et al., 2016). In addition, such approaches cannot detect potential disease-causing cryptic micro-deletions or microduplications (Vermeesch et al., 2016).

The technique of mate-pair next-generation sequencing is also labor intensive, and while it can delineate chromosomal structural rearrangements with abnormal phenotypes, it may not perform well when the breakpoint is in a highly repetitive genomic region (Aristidou et al., 2017).

This example illustrates that embryos can be accurately distinguished to show which embryos carry a BCR and which embryos do not. Further, it is possible to detect the presence of cryptic imbalances and complex rearrangements, using PGT-SR with the MINION™ long-read nanopore sequencer (Oxford Nanopore Technologies).

Methods & Results

Patient Samples

This study was approved by the University of Toronto Research Ethics Board (REB no. 30251) and informed consent was obtained from all participants. Eleven couples, with one of the partners being a carrier of a balanced chromosomal rearrangement (BCR), underwent IVF at the CReATe Fertility Centre, Toronto, Canada from 2016-18. Nine were undergoing IVF with PGT-SR due to known BCR (7 reciprocal translocations, 1 pericentric inversion, 1 paracentric inversion), and 2 were referred for IVF-PGT-A testing where the male was diagnosed with a balanced translocation after detection of BCR indicative chromosomal aberrations in their embryos. Ten couples completed one IVF cycle after recruitment and had all blastocysts biopsied for PGT-SR. Two couples completed more than one IVF cycle and WGA DNA was batched from all of their embryos and tested together.

Confirmatory postnatal testing on 5 children born to date was performed. From each couple and/or affected family member whole blood collected with EDTA was used for DNA extraction while from patient carrier of BCR peripheral blood mononuclear cells were collected for evaluation of allele drop out (ADO) frequency for the established cBP-PCR in WGA samples.

Embryonic DNA Samples

Embryos were created through in vitro fertilization (IVF) of mature (metaphase II) oocytes by intracytoplasmic sperm injection (ICSI). After ICSI, the fertilized oocytes were cultured individually from Day 1 (D1) to D5/6 in Sage 1-Step medium with serum protein supplement (Origio, Canada) under oil, in 25-μL droplets. Trophectoderm (TE) biopsy of 4-6 cells was performed on a total of 82 blastocysts from the 11 couples using laser-assistance performed in Global “total” w/Hepes and protein medium (LifeGlobal, Canada) under oil. All embryos were cryopreserved by vitrification after laser collapse of the blastocoel cavity, as per standard procedures (Darwish et al., 2016).

WGA using SUREPLEX™ system (Illumina, CA) or the REPLI-G™ kit (QIAGen, Germany) using the manufacturer's protocols, was performed on the TE biopsy cells. Embryonic WGA DNA was quantified by QUBIT™ instrumentation and examined for size distribution on a 1% e-Gel.

Standard PGT-SR short read NGS based testing was performed using the VERISEQ™-PGS kit (Illumina, CA).

Study Design

A workflow for comprehensive PGT-SR to distinguish embryos carrying a BCR from non-carriers at single-base resolution was developed and clinically validated with the MINION™ sequencer (long read sequencing) (Oxford Nanopore Technologies ONT, UK) that streamlined implementation within the current clinical PGT-SR (short read sequencing) diagnostic processes. The target timeline to result delivery from the comprehensive PGT-SR testing was within 7 days of embryo biopsy.

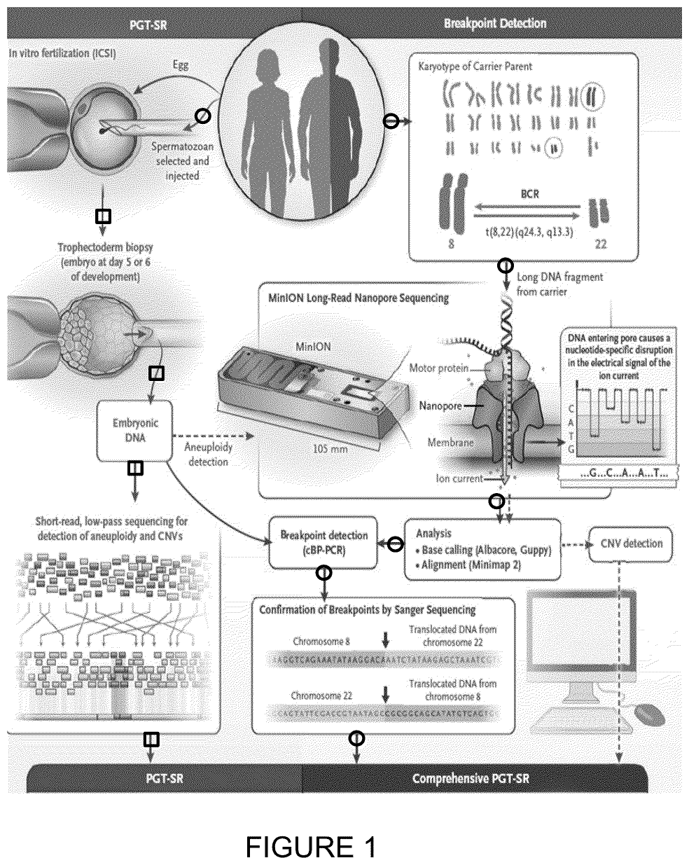

FIG. 1 provides a schematic representation of preimplantation genetic testing for structural rearrangements with the use of Long-Read Nanopore Sequencing, as described herein. Arrows marked with a circle indicate the approach used for the delineation of balanced translocations and inversions after long-read DNA sequencing is performed with the MINION™ nanopore sequencer (Oxford Nanopore Technologies). Such delineation allows for breakpoint confirmation by means of a polymerase-chain-reaction (PCR) assay customized to the breakpoint (cBP-PCR), together with single-base resolution of the breakpoint by means of Sanger sequencing. High-molecular-weight DNA extracted from balanced chromosomal rearrangement (BCR) carriers was fragmented, and libraries were prepared for long-read sequencing with the use of the SQK-LSK108 ligation kit (Oxford Nanopore Technologies). Long-read sequencing was performed on the MINION™ sequencer over 48 hours.

The average length of the sequenced fragments was approximately 8 to 10 kb, with the longest reads reaching over 100 kb. Karyotype-guided computational analysis identified derivative reads and mapped breakpoints. The use of custom-designed PCR primers and Sanger sequencing yielded confirmation of breakpoints within 7 days after the sample was submitted.

The arrows marked with a square indicate the approach used for preimplantation testing for structural rearrangements (PGT-SR) with short-read sequencing, which can detect only chromosomal gains and losses (and not BCRs) in embryos. Cells from in vitro fertilized embryos of couples undergoing PGT-SR were biopsied on day 5 or 6 of development (before vitrification) to obtain DNA for aneuploidy (chromosomal copy-number variation [CNV]) detection and for the discrimination of embryos that do not carry a BCR from those that do with the use of the custom breakpoint PCR designed for the BCR-carrier parent.

The dashed arrows indicate an approach in which embryonic DNA obtained from the same trophectoderm biopsy sample can also be used for aneuploidy detection by means of sequencing with the MINION™ sequencer and an algorithm for CNV analysis. ICSI denotes intracytoplasmic sperm injection.

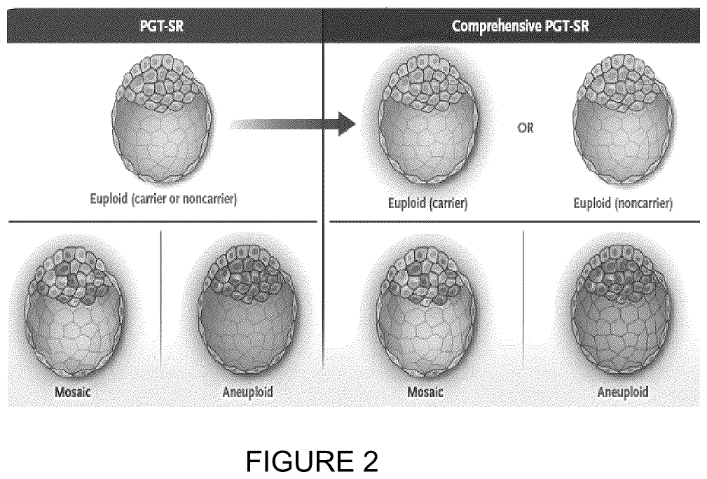

FIG. 2 shows a schematic of the evaluation of embryos from FIG. 1 in regard to preimplantation genetic testing for structural rearrangements (PGT-SR) when comprehensive PGT-SR, is used to differentiate with regard to Euploid (carrier), Euploid (noncarrier), mosaic and aneuploid embryos.

Localization and determination of orientation of the two chromosomal breakpoints was conducted using low coverage sequencing of the carrier partner with the MINION™ sequencer and analysis guided by the karyotype (determined by previous cytogenetic testing). With this information custom PCR primers were designed to allow for breakpoint amplification and confirmation using Sanger sequencing. Next, this custom designed breakpoint PCR (cBP-PCR) was used for pre-implantation testing on whole genome amplified (WGA) DNA from embryos. After amplification, Sanger sequencing achieved single base resolution of the rearrangement. Aneuploidy testing (PGT-A) on WGA DNA from embryos was performed with standard PGT-A VERISEQ™ NGS technology (Illumina, CA) on a MISEQ™ system (Illumina, CA). Copy number analysis for aneuploidy testing was also performed using DNA ligation kit on MINION™ sequencer. SNP array Karyomapping (Illumina, CA) was also used to confirm findings from data generated using the MINION™ sequencer in the cases where DNA from an affected child or embryo with an unbalanced chromosomal rearrangement was available for haplotyping (Wang et al, 2017; Handyside et al., 2010).

Table 1 provides information illustrating that balanced structural rearrangements (BCR) in patients and their IVF created embryos undergoing comprehensive PGT-SR to distinguish euploid BCR-carrier from non-carrier embryos. Parental karyotype guided analysis of nanopore sequencing data from the MINION™ sequencer determined the genomic location of the breakpoint utilized to diagnose embryos BCR carriers.

TABLE 1
Balanced structural rearrangements (BCR) in patients and their IVF created embryos

| | | Nanopore | | | | | |
|---|---|---|---|---|---|---|---|
| | Chromosomal | Breakpoint | Total | Unbalanced/ | Euploid/ | | |
| | rearrangement | Detection (Hg38) | biopsied | aneuploid | BCR | | BCR |
| Case | (Karyotype) | (Orientation) | blastocysts | embryos | carriers^{$} | Euploid^{#} | carriers^{#} |
| TC-1 | 46, XX, | chr8: 139353588 (+); | 4 | 1 | 3 | 2 | 1 |
| | t(8; 22) | chr22: 46852062 (+) | | | | | |
| | (q24.3; | | | | | | |
| | q11.2) | | | | | | |
| TC-2 | 46, XX, | chr1: 46843185 (+); | 14* | 11 | 3 | 1 | 2 |
| | t(1, 2) | chr2: 71426279 (+) | | | | | |
| | (p34.1; | | | | | | |
| | p13) | | | | | | |
| TC-3 | 46, XX, | chr6: 3042566 (+); | 5 | 2 | 3 | 2 | 1 |
| | t(6, 14) | chr14: 64741771 (−) | | | | | |
| | (p25; | | | | | | |
| | q23.3) | | | | | | |
| TC-4 | 46, XY, | Chr4p16.3: 49256596 (−); | 4 | 2 | 2 | 0 | 2 |
| | inv(4) | chr4q32.2: 160864117(+) | | | | | |
| | (p16.3; | | | | | | |
| | q32.3) | | | | | | |
| TC-5 | 46, XX, | chr1: 7789560 (+); | | | | | |
| | t(1:22) | chr22: 48969135 (−) | | | | | |
| | (p36.2; | | | | | | |
| | q13.3) | | | | | | |
| TC-6 | 46, XX, | chr8q13: 69999154(+); | 3 | 1 | 2 | 1 | 1 |
| | inv (8) | chr8q24.3: 142849210(−) | | | | | |
| | (q13; | | | | | | |
| | q24.3) | | | | | | |
| TC-7 | 46, XX, | chr11: 116812508(+); | 3 | 3 | 0 | 0 | 0 |
| | t(11: 22) | chr22: 20339429(+) | | | | | |
| | (q23.2; | | | | | | |
| | q11.2) | | | | | | |
| TC-8 | 46, XX, | chr11: 124203274(+); | 25** | 19 | 6 | 3 | 3 |
| | t(11: 12) | chr12: 67201427 (+) | | | | | |
| | (11q24.2; | | | | | | |
| | q12q14.3) | | | | | | |
| TC-9\hat{ } | 46, XY, | chr8: 124483128(+); | 6 | 4 | 2 | 1 | 1 |
| | t(8: 11) | chr11: 116814391(+) | | | | | |
| | (q24.13, | | | | | | |
| | q23.3) | | | | | | |
| TC-10 | 46, XY, | chr8: 137676929(+); | 5 | 2 | 3 | 1 | 2 |
| | t(8; 12) | chr12: 73058076(+) | | | | | |
| | (q24.22; | | | | | | |
| | q14) | | | | | | |
| TC-11\hat{ } | 46, XY, | chr1: 57376472(+); | 13 | 10 | 3 | 1 | 2 |
| | t(1; 6) | chr6: 16984092(+) | | | | | |
| | (p32.2; | | | | | | |
| | p22.3) | | | | | | |

^{$}Standard short read sequencing PGT-SR utilizes low pass (0.1x coverage, short read) whole genome sequencing and has inherent limitation to distinguish euploid embryos BCR carriers from non-carriers
^{#}Nanopore long read sequencing PGT-SR enables discrimination of euploid BCR carriers from non-carriers embryos by accurate delineation of the chromosomal rearrangement in parents BCR carriers
*Embryos from 3 IVF cycles (3OPU);
**Embryos from 5 IVF cycles (5OPU)
\hat{ }Incidental finding of balanced translocation in the parent. Preimplantation testing of their embryos revealed high rate of unbalanced aberrations indicative for BCR carriers. MinION ™ nanopore sequencing determined the breakpoints that were independently confirmed by karyotype and FISH analysis of the parent.

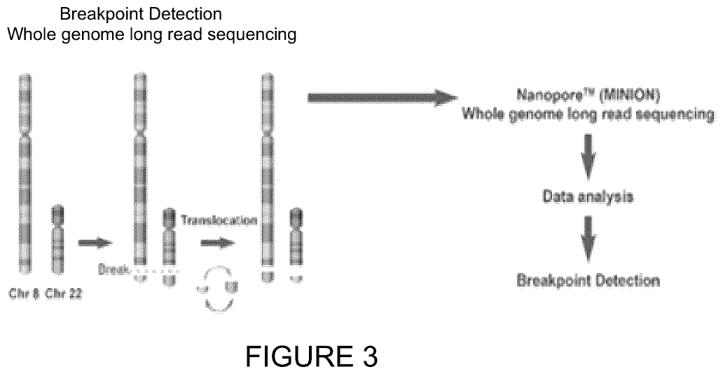

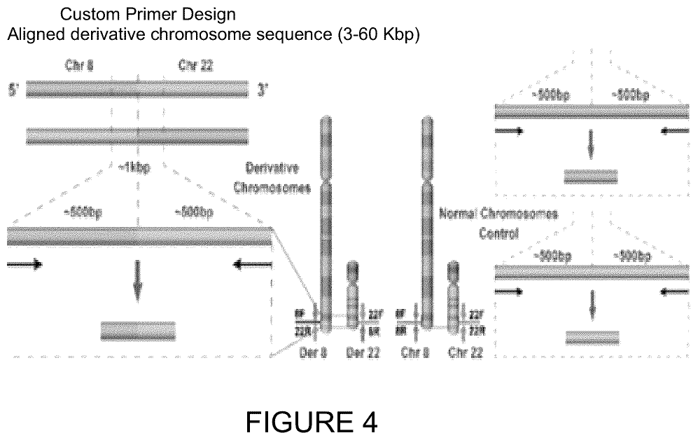

FIG. 3 and FIG. 4 represent the establishment of nanopore sequencing strategy for detection of breakpoints in BCR carriers. In FIG. 3, using the nanopore MINION™ sequencer, the breakpoints were determined in the parent, TC-1 (46,XX,t(8:22)(q24.3,q13.33), and affected child (22q13 deletion syndrome, [arr[hg19]:8q24.3 (140,366,315-146,295,771)×3, 22q13.31q13.33 (47,254,703-51,176,099)×1]) on both chromosomes at ˜4× genome coverage, producing 5 reads (6 kb±560 bp) spanning the breakpoints of derivative chromosomes. The breakpoint locations were consistent with the karyotyping coordinates but were ˜500 kb and ˜2 Mb upstream from the breakpoints identified for chr8 and for chr 22 with SNP array, respectively. The breakpoints and corresponding normal chromosomes were amplified (using custom primers cBP-PCR), as schematically represented in FIG. 4.

Sanger sequencing was used to identify the breakpoint location to a single base resolution. Single base resolution (a breakpoint for balanced carrier) is located: Chr 8—139353389-139353718 (breakpoint 139,353,588), and Chr 22—46851362-46852103 (breakpoint 46,852,062). The primers were validated and tested for robustness against the balanced carrier (TC-1), affected child (TC-1_P), non-carrier DNA (TC-1_1-father) and on low template WGA (a sample of 5 single lymphocytes from the mother) (TC-1_SC), correctly determining the carrier status.

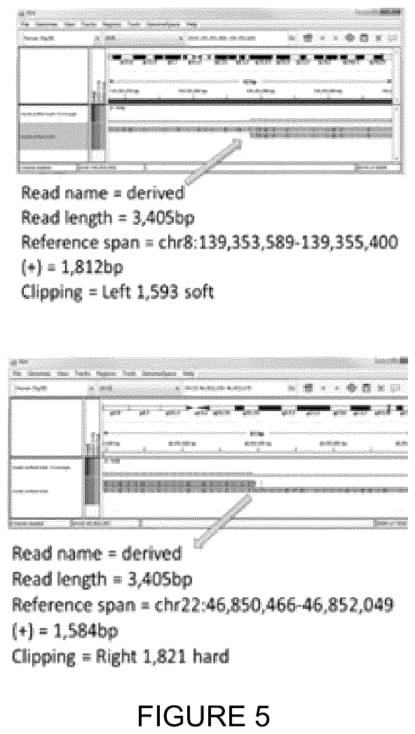

FIG. 5 provides computer screen shots of data pertaining to FIG. 3 and FIG. 4 with regard to Chr 8 (upper panel) and Chr 22 (lower panel), respectively:
Read name=derived, Read length=3,405 bp, Reference span=chr8:139,353,589-139,355,400, (+)=1,812 pb, Clipping=Left 1,593 soft; and
Read name=derived, Read length=3,405 bp, Reference span=chr22:46,850,466-46,852,049, (+)=1,584 pb, Clipping=Right 1,821 hard.

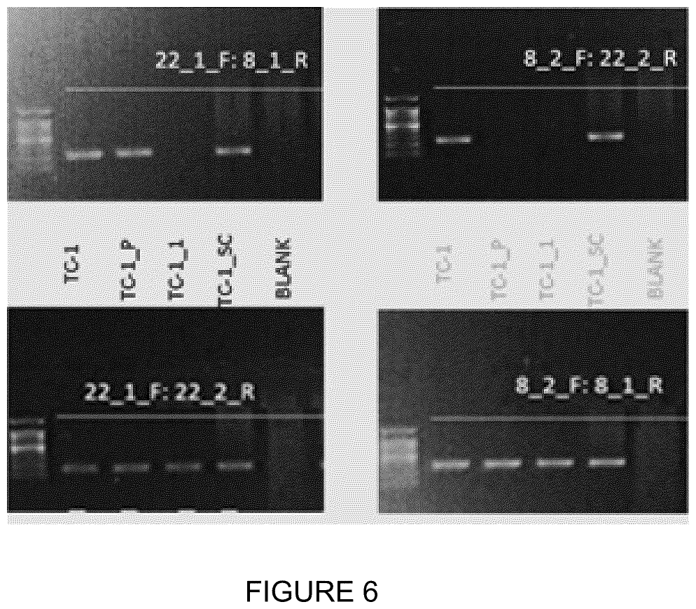

FIG. 6 S1R-L illustrates chromatographic evaluation pertaining to 22_1_F:8_1_R and 8_2_F:22_2_R for the upper panels, respectively; and 22_1_F:22_2_R and 8_2_F:8_1_R for the lower panels, respectively.

Long Read Nanopore Sequencing with MINION™ Sequencer.

Genomic DNA was extracted from peripheral blood from each partner and affected or unaffected children (if available) following a modified Phenol/Chloroform protocol to obtain high molecular weight DNA (hmwDNA) from 4 mL (EDTA) peripheral blood. Briefly after red cell lysis (1×Pharm lyse buffer, DB Biosciences), white blood cells were lysed in Proteinase K/SDS (0.5% w/v) TRIS-HCl buffer; and protein precipitation and DNA extraction were performed using standard Phenol/Phenol-Chlorophorm-Isoamylalchohol DNA extraction protocols. The quality and integrity of the DNA was measured by NANODROP™ spectrophotometer and QUIBIT™ fluorometer 2.0 (Life Sciences).

1 μg of hmwDNA was sheared to ˜10-20 kb fragments using G-tubes (Covaris). Genomic libraries were prepared using the Oxford Nanopore 1D-ligation library prep kit SQK-LSK108 following ONT protocol (Oxford Nanopore Technologies, UK). A 0.4× (instead of 1×) AMPURE™ magnetic beads (Beckman Coulter, A63881) clean-up was introduced after the end-repair steps (NEBNext Ultra II end-repair/dA-tailing module (New England BioLabs; E7546)) in the protocol to remove small DNA fragments. For 1D library the ligation reaction was prepared as follows: 30 μL of water (DNA), 20 μL of adaptor mix and 50 μL of blunt/TA ligase master mix (New England BioLabs; M0367).

Ligation was carried out at room temperature for 15 min. The AMPURE™ XP beads were re-suspended at room temperature by vortexing. 40 μL of beads were added into the ligation product. The beads were washed twice with 140 μL of adaptor bead binding buffer and eluted in 25 μL of elution buffer. The eluted product was the adaptor-ligated library that was prepared for sequencing using the pre-sequencing mix and loading beads used in nanopore sequencing. Each genomic library was loaded and sequenced on a R9.4 flowcell supported by MINKNOW™ software v 2.13 (Oxford Nanopore Technologies, UK, herein “ONT”) for 48 hours on a MINION™ sequencer using MINKNOW™ software v 2.13 (ONT). The total technician hands-on time for library preparation was 30 min with 2 hours overall time.

Nanopore Sequencing Data Analysis for Detection of Breakpoints.

An analytical pipeline was developed for breakpoint detection which involves base calling, alignment, data quality filtering, local search and extraction of reads aligning to candidate BCR regions determined by karyotyping in all patients, followed by visual inspection. Basecalls were generated from the MINION™ sequencer data using ALBACORE™ (version 2.2.7). Reads passing the basecaller's quality filter (mean quality score ≥7) were aligned to the hg38 human reference with minimap2, version 2.1-r311 (Li, 2018). Each sample's run-level alignments were merged into a single per sample alignment (BAM format) using samtools 6 (Li, 2009). For suspected genomic rearrangements, karyotype coordinates were converted to genomic coordinates using the UCSC hg38 genome build, and reads that aligned to both of the regions were extracted. A local search was done for supporting reads in the aligned MINION™ sequencer data followed by visual inspection with the IGV genome browser. This analysis pipeline allowed confirmation of breakpoints for abnormalities previously discovered through cytogenetics with a mean genome coverage of 3.5×, and an average read length of 4 kb (see Table 2).

After identification of the breakpoint junctions and orientation of the rearranged fragments, flanking DNA sequences 500 bp upstream from the breakpoints were extracted to create a cBP-PCR system enabling amplification over each breakpoint's flanking region and the respective regions of the two normal chromosomes (Untergasser et al., 2012). All cBP-PCR products from parental DNA and embryo WGA DNA samples were also analyzed by Sanger sequencing (3500 Genetic Analyzer, Applied Biosystems) using BigDye™ v3.1 to confirm the breakpoint and non-breakpoint flanking sequences and provide single-base resolution of the breakpoint.

Table 2 shows long-read nanopore sequencing metrics presenting the yield, quality of sequencing and diagnostic yield of split reads per patient. Raw coverage is calculated from sequencing yield and a genome size of 3.3 Gb.

TABLE 2
Long-read nanopore sequencing metrics

| | | | | | Raw | |
|---|---|---|---|---|---|---|
| | No of | Number of | Number of | Average read | Coverage | Number of putative |
| Sample | Flowcells | reads | bases (GB) | length (Bp) | (X) | breakpoint reads |
| TC-1 | 4 | 967,849 | 10.6 | 10952.12 | 3.21 | 5 |
| TC-2 | 3 | 885,617 | 9.1 | 10275.32 | 2.76 | 6 |
| TC-3 | 6 | 2,393,639 | 21.3 | 8898.58 | 6.45 | 4 |
| TC-4 | 4 | 1,165,964 | 13.5 | 11578.40 | 4.09 | 2 |
| TC-5 | 3 | 934,209 | 10.1 | 10811.29 | 3.06 | 3 |
| TC-6 | 1 | 823,265 | 6.69 | 8126.18 | 2.03 | 6 |
| TC-7 | 2 | 1,803,316 | 13.17 | 7303.21 | 3.99 | 2 |
| TC-8 | 2 | 1,416,138 | 12.17 | 8593.80 | 3.69 | 3 |
| TC-9 | 7 | 6,245,003 | 16.65 | 2665.73 | 5.05 | 2 |
| TC-10 | 2 | 3,232,042 | 23.91 | 7397.80 | 7.25 | 6 |
| TC-11 | 1 | 3,686,153 | 16.43 | 4457.75 | 4.98 | 4 |

Nanopore Library Preparation and Sequencing of Embryonic WGA DNA.

Whole genome sequencing (WGS) using the MINION™ sequencer was performed on 6 WGA embryonic DNA samples. 1 μg of WGA DNA was first repaired using T7-exonuclease (New England BioLabs, UK) to remove DNA branching, then sheared using G-tubes (Covaris). The same 1D-ligation library preparation kit (SQK-LSK108) described above was used as for genomic DNA. Libraries from each embryo were loaded and sequenced on a R9.4 flowcell supported by MinKnow™ software v 2.13 (ONT) for 48 hours on the MINION™ sequencer. Additionally, we developed an analytical pipeline to detect copy number variations in WGA DNA from embryos and provide PGT-A result based on nanopore sequencing.

Detection of Copy Number Variants (CNV) in Embryo WGA Using Nanopore Sequencing.

Bias in WGA leads to the artificial inflation of read counts and poses a challenge to analyse copy number variations (CNV). An open-source tool Ginkgo™, was used to address these issues and detect CNVs with low coverage sequencing (Garvin et al., 2015). Aligned reads were binned into 1 Mb variable-length intervals across the genome to assess GC bias and coverage dispersion. Cohorts TC-1, TC-2 and TC-3 were used as a reference to normalize bin counts and to eliminate biases uncorrected by GC normalization (Wei et al., 2018). Copy-number profiles of individual biopsies were constructed with a better signal-to-noise ratios, and was reliable for accurate copy-number calls.

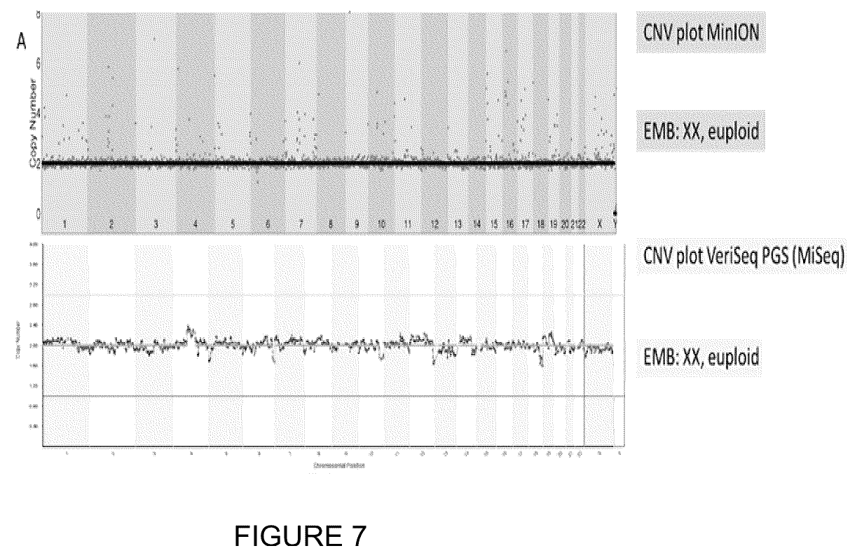

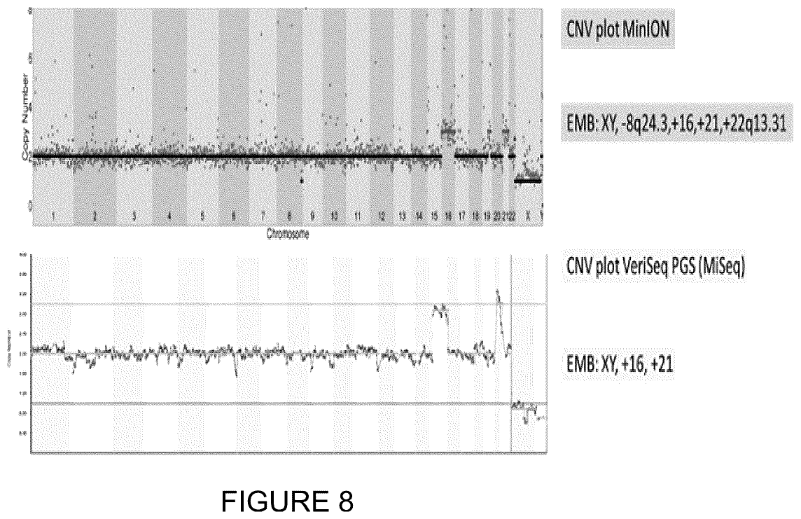

FIG. 7 and FIG. 8 show the detection of chromosomal CNV (aneuploidy) in embryos using the MINION™ sequencer. CNV plots generated using the MINION™ sequencer for whole genome sequencing data compared to standard short read sequencing using VERISEQ™ PGS kit on the MISEQ™ system. The X axis on each plot represents chromosomes 1-22, X and Y; Y axis represents copy number of each region, and gains and losses are indicated by dots. FIG. 7 provides a CNV plot presenting a euploid, XX embryo (EMB-2, TC-1) and FIG. 8 illustrates aneuploidy, XY embryo with trisomy 16 and 21 and in addition it detected the del/dup associated with unbalanced translocation del8q24.3, dup22q13.31 (EMB-6 from patient TC-1).

Table 3 summarizes the concordance between the CNV result of the MINION™ sequencer and the VERISEQ™ PGS kit result. Eight embryos were sequenced with the MINION™ sequencer, as a proof of principle, and CNV detection using the method described above was compared to the clinical PGT-A (VERISEQ™ PGS Kit). The close concordance of the results validates this methodology.

TABLE 3
Long-read nanopore sequencing CNV detection in embryos

| | MINION ™ sequencer | VERISEQ ™ |
|---|---|---|
| Sample | CNV result | PGS result |
| EMB-2, TC-1 | 46, XX | 46, XX |
| EMB-3, TC-1 | 46, XX | 46, XX |
| EMB-5, TC-1 | 46, XX | 46, XX |
| EMB-6, TC-1 | 48, XY: +16; +21; −8q24.3 | 48, XY: +16; +21 |
| | (6.9Mb); +22q13.31 (4.3Mb) | |
| EMB-4, TC-3 | 46, XY | 46, XY |
| EMB-6, TC-3 | 46, XY | 46, XY |
| EMB-1, TC-4 | 46, XX | 46, XX |
| EMB-1, TC-8 | 45, XX, −16 | 45, XX, −16 |

Design and Validation of Custom Breakpoint Amplification System—cBP-PCR.

Bioinformatics analysis of the MINION™ sequencer data identified candidate breakpoints from reads that mapped to derivative chromosomes from the BCR. The analysis provided the breakpoint location within 100 bp (up or downstream) of the actual breakpoint. After identification of the approximate breakpoint junctions and the orientation of the rearranged fragments, flanking DNA sequences 500 bp upstream were extracted. Primer3 (Untergasser et al., 2012) was used to design PCR primers using these flanking sequences. Candidate primers were screened for off-target amplification using the UCSC Genome Browser's in-silico PCR tool. The candidate diagnostic PCR (cBP-PCR) system consisted of two sets of primers used in four different combinations, allowing for amplification over each breakpoint's flanking region and the respective regions of the two normal chromosomes.

For the affected chromosomes, amplicons were designed either around contacting sequences from the 2 chromosomes involved in the translocation or 2 distant chromosomal regions in cases of inversion. In cases where the translocated strand was in an opposite orientation, 2 additional primer pairs were designed to amplify the normal chromosome in the region corresponding to the one flanking the breakpoints. Primers were tested and validated against 10 ng of carrier and non-carrier parental DNA, as well as 50 ng of WGA DNA from all embryos (see FIG. 3 to FIG. 6).

PCR reactions were carried out in a total volume of 25 μL using QIAGen PCR Master mix and 100 pmol of each primer. Primers were designed to have T, of 60° C. and standard cycling times for fragments between 250-750 bp. All PCR products derived from parental DNA and embryo WGA DNA samples were analyzed by Sanger sequencing (3500 Genetic Analyzer, Applied Biosystems) using BigDye v3.1 to confirm the breakpoint and non-breakpoint flanking sequences and provide breakpoint single base resolution.

Allele Drop Out (ADO) Testing Using Parental Mononuclear Cells.

Peripheral blood mononuclear cells (PBMCs) were collected by gradient centrifugation of 4 mL (EDTA) blood from TC-1 and used for evaluation of allele drop out (ADO) frequency for the established CDB-PCR in WGA samples. In brief, the blood was diluted 4× with phosphate buffered saline (PBS). Subsequently 13 mL of Histopaque®-1077 (family 1; Sigma-Aldrich 10771-500ML) was added per 35 mL of diluted blood. The resulting mixture was centrifuged at room temperature for 20 minutes at 900×g, followed by recovery of the PBMC layer. PBMC were washed twice using PBS, centrifuged at 750×g for 5 minutes, and re-suspended in PBS with 50% DMSO. Low template samples were created by WGA of 5-6 PBMC using SUREPLEX™ (Illumina, CA), following the manufacturer's suggested protocol.

Standard PGT-SR Using Short Read NGS.

Whole genome copy number variation (CNV) analysis was performed by whole genome low pass (0.1×) NGS, using the VERISEQ™ PGS Kit (Illumina, CA). Briefly, after WGA according to manufacturer's instructions, gDNA was tagmented and amplified. The amplified DNA was indexed, purified using AMPure XP beads (1:1 ratio), and normalized with magnetic beads. Normalized libraries were pooled, denatured, and sequenced using a MISEQ™ system (single-end, 1×36 bp). BLUEFUSE MULTI™ (Illumina, CA) was used for chromosome CNV analysis and data visualization. The optimal metrics for PGS are 500,000 reads passing filter and a sample noise score (Derivative Log Ratio—DLR) of <0.2; 250,000 reads and DLR<0.4 is clinically acceptable. Reporting resolution is 10 Mb in size for copy number aberrations and >30% for of mosaicism level.

Repeat Analysis

RepeatMasker (www.repeatmasker.org) determined whether the breakpoints fall in regions containing repetitive sequences and, if so, the class of repeats. DNA sequences from split reads spanning the breakpoint were extracted from the nanopore read and annotated to different repeat classes by alignment with public annotated repeat sequences using RepeatMasker. The proximity of the breakpoint to coding/non-coding genes was estimated and is presented in Table 4 for TC-1 to TC-6 and Table 5 for TC-7 to TC-11.

TABLE 4
Summary of repeat elements 1 kb 5′ and 3′ of the breakpoints
determined by MinION sequencing (TC-1 to TC-6)

| | TC-1 | | TC-2 | | TC-3 | | TC-4 | | TC-5 | | TC-6 | |
|---|---|---|---|---|---|---|---|---|---|---|---|---|
| | t(8; 22) | | t(1, 2) | | t(6, 14) | | Inv (4) | | t(1: 22) | | Inv (8) | |
| | q24.3; | | p34.1; | | p25; | | p16.3; | | p36.2; | | q13; | |
| | q11.2 | | p13 | | q23.3 | | q32.3 | | q13.3 | | q24.3 | |
| Repeat Class | chr 8 | chr 22 | chr 1 | chr 2 | chr 6 | chr 14 | chr 4 | chr 4 | chr 1 | chr 22 | chr 8 | chr 8 |
| DNA | | | | | | | | | | | 1 | |
| LINE | 2 | 1 | 1 | | 2 | | 3 | 3 | 3 | 1 | | 3 |
| LTR | | | 1 | | 3 | | | 3 | 3 | 1 | | |
| SINE | 1 | | 1 | 1 | 2 | | 1 | 1 | 1 | | 4 | 1 |
| Satellite | | | | | | | | | | | | |
| Simple | | | | | | | | | 1 | | | 1 |
| Repeat | | | | | | | | | | | | |
| Repeat | chr 8 | chr 22 | chr 1 | chr 2 | chr 6 | chr 14 | chr 4 | chr 4 | chr 1 | chr 22 | chr 8 | chr 8 |
| Families | | | | | | | | | | | | |
| Alu | 1 | | 1 | 1 | 2 | | 1 | 1 | 1 | | 3 | 1 |
| CR1 | | | | | | | | | | | | |
| ERV1 | | | | | 3 | | | | | | | |
| ERVL | | | | | | | | | | 1 | | |
| ERVL-MaLR | | | 1 | | | | | 3 | 3 | | | |
| L1 | 2 | 1 | 1 | | 2 | | 3 | 3 | 2 | 1 | | 3 |
| L2 | | | | | | | | | | | | |
| MIR | | | | | | | | | 1 | | 1 | |
| Satellite | | | | | | | | | | | | |
| Simple | | | | | | | | | 1 | | | 1 |
| Repeat | | | | | | | | | | | | |
| hAT-Blackjack | | | | | | | | | | | 1 | |
| hAT-Charlie | | | | | | | | | | | | |

TABLE 5
Summary of repeat elements 1 kb 5′ and 3′ of the breakpoints
determined by MinION sequencing (TC-7 to TC-11)

| | TC-6 | | TC-7 | | TC-8 | | TC-9 | | TC-10 | | TC-11 | |
|---|---|---|---|---|---|---|---|---|---|---|---|---|
| | Inv (8) | | t(11:22) | | t(11:12) | | t(8:11) | | t(8; 12) | | t(1; 6) | |
| | q13; | | q23.2; | | 11q24.2, | | q24.13, | | q24.22; | | p32.2; | |
| | q24.3 | | q11.2 | | q12q14.3 | | q23.3 | | q14 | | p22.3 | |
| Repeat Class | chr8 | chr8 | chr11 | chr22 | chr11 | chr12 | chr8 | chr11 | chr8 | chr12 | chr1 | chr6 |
| DNA | 1 | | | | | | | | | 1 | 1 | |
| LINE | | 3 | 5 | | 3 | 1 | 1 | 2 | 1 | 2 | 4 | 1 |
| LTR | | | 1 | | | | | 1 | 1 | 2 | 2 | 1 |
| SINE | 4 | 1 | 2 | 3 | | 2 | 2 | 2 | | 2 | | |
| Satellite | | | | 1 | | | | | | | | |
| Simple | | 1 | 1 | 2 | | | 1 | | | | | |
| Repeat | | | | | | | | | | | | |
| Repeat Families | chr8 | chr8 | chr11 | chr22 | chr11 | chr12 | chr8 | chr11 | chr8 | chr12 | chr1 | chr6 |
| Alu | 3 | 1 | 2 | 3 | | 1 | 2 | 2 | | 2 | 1 | 1 |
| CR1 | | | 1 | | | 1 | | | | | | |
| ERV1 | | | | | | | | | | | | |
| ERVL | | | | | | | | | | | | |
| ERVL-MaLR | | | 1 | | | | | 1 | 1 | 2 | | |
| L1 | | 3 | 2 | | 3 | | | 2 | 1 | 2 | | |
| L2 | | | 2 | | | | 1 | | | | 4 | 1 |
| MIR | 1 | | | | | 1 | | | | | 1 | |
| Satellite | | | | 1 | | | | | | | | |
| Simple | | 1 | | | | | 1 | | | | | |
| Repeat | | | | | | | | | | | | |
| hAT-Blackjack | 1 | | | | | | | | | | | |
| hAT-Charlie | | | 1 | 2 | | | | | | 1 | 1 | |

SNP Array Testing for Pre-Implantation Haplotyping.

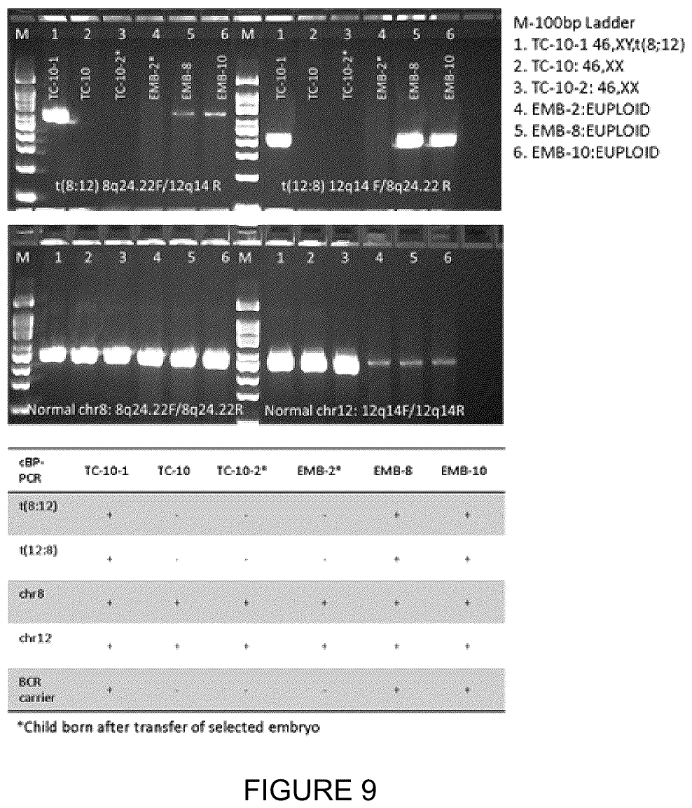

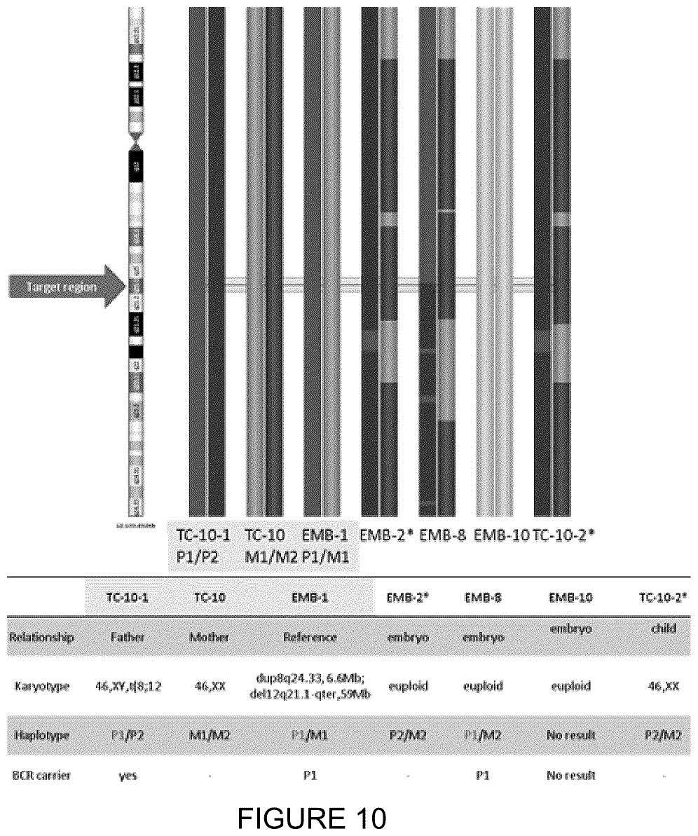

SNP based genotyping was performed with the Infinium Karyomapping Assay that utilizes the Illumina Human Karyomap-12 V1.0 microarray, as previously described (Handyside et al., 2010). Following manufacturer's protocol technician hands-on time was over 6 hours for a total protocol time of 2 days. Each Karyomap-12 bead chip contained approximately 300,000 SNPs. The analysis was performed using BlueFuse software (Illumina, CA) based on informative SNPs in the breakpoint region. SNPs are informative if they are heterozygous in the carrier and homozygous in the partner and should be homozygous in the carrier's parents or other family members. This information was used to determine the carrier's haplotype linked to the derivative chromosome or to the normal chromosome. An affected child or an unbalanced SR embryo was used as a reference. The haplotypes of the whole chromosomes involved in the balanced translocation and the corresponding normal homologous chromosomes could indicate the presence of homologous recombination around the breakpoints. The benchmark to distinguish balanced SR vs. normal embryos combined haplotypes of the breakpoint regions and the presence of homologous recombination in these regions. When the embryo's haplotype was the same as the unbalanced or affected reference samples (in-phase with reference), it was defined as a balanced translocation carrier (FIG. 9 and FIG. 10). However, if the embryo haplotype differed (out-of-phase) from the unbalanced/affected reference sample, it was defined as non-carrier. Prediction of carrier status of an embryo is limited to cases free of recombination in proximity to the breakpoints 10-12.

FIG. 9 and FIG. 10 show concordant results of pre-implantation balanced translocation carrier determination by nanopore sequencing with a MINION™ sequencer and SNP haplotyping. In FIG. 9, results of a custom designed breakpoint PCR using established MINION™ sequencer breakpoint mapping strategy is illustrated. Breakpoint amplification is present in the parent (TC-10-1) and embryos balanced translocation carriers (EMB-8 and 10), and absent in the parent (TC-10), embryo EMB-2 and child born after transferring this embryo (TC-10-2). In FIG. 10, SNP haplotyping using Karyomapping Infinium Arrays visualized using BlueFuse multi software is illustrated. Unbalanced embryo from the same IVF cycle was used as reference for haplotype phasing. Paternal haplotype P1 is associated with the rearranged chromosomes. Arrow indicates the region of interest that was karyotype guided. EMB-2 inherited normal paternal allele (P2) corresponds to the haplotype and karyotype of the child born after transfer of this embryo.

Nanopore Long-Read Sequencing can Precisely Determine Breakpoints in Balanced Rearrangements.

Nine known balanced translocation and inversion carriers and 2 suspected balanced translocation carriers were sequenced using our nanopore sequencing cBP-PCR analysis pipeline. Between 2 and 8 derivative reads spanned the breakpoints, with an average read length of 4.5 kb (metrics presented in Table 2). Breakpoints were successfully identified in all cases. The long-read sequencing enabled the determination of the strand orientation after translocation/inversion, which is essential to design the cBP-PCR primers. Table 1 summarizes the location of each rearrangement determined by karyotyping and by long-read sequencing. For all patients, the breakpoints detected by long-read sequencing were within the cytogenetic bands determined by karyotyping. Genomic coordinates of the translocations detected in the two cases with incidental BCR findings (TC9 and TC11) were first identified by sequencing with the MINION™ sequencer and later confirmed by conventional karyotyping and FISH analysis of the parent.

All 22 translocation/inversion breakpoints studied, were located within repeated elements (Table 4 and Table 5).

Table 6 and Table 7 summarize the genes upstream and downstream of the breakpoints. Only one breakpoint (TC11, chr 1) disrupted a gene, DAB1, associated with Spinocerebellar ataxia 37 and Lissencephaly with cerebellar hypoplasia, described in details below.

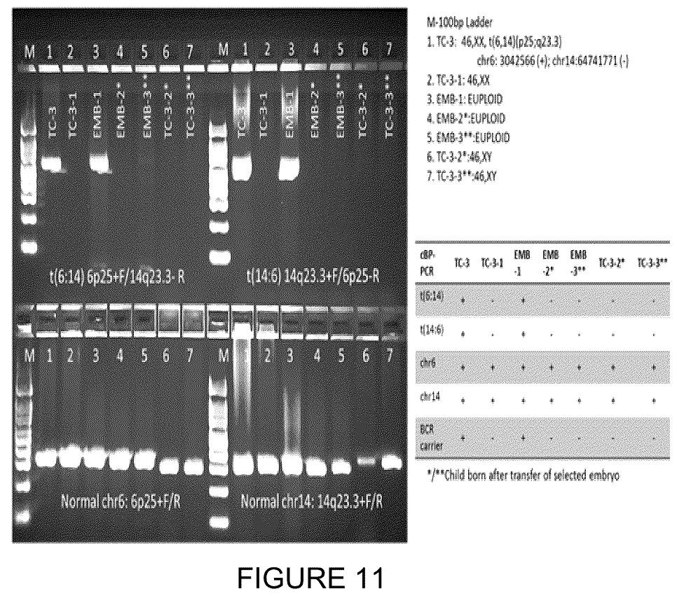

Sanger sequencing determined the flanking regions of the breakpoint and detected an insertion of a sequence at the breakpoint junctions. Results from the cBP-PCR and Sanger sequencing are shown in FIG. 11 to FIG. 14. Sanger sequencing of the breakpoint junctions revealed small insertions/deletions (1-5 bases) in TC3, TC9, and TC11. Remaining translocations and the two inversions were fully balanced. Each cBP-PCR set was robust and reproducible without allele dropout when tested on low template lymphocyte DNA (5 cell sample) from BCR carriers.

Table 6 and Table 7 show genes within 100 kb of breakpoint sites assessed using USCS genome browser and genomic coordinates determined by sequencing with a™ sequencer of patients carriers of balanced rearrangement, for TC-1 to TC-6 (Table 6) and for TC-6 to TC-11 (Table 7). Gene density within 100 kb of the 22 breakpoints analyzed in the 11 BCR carriers is presented, with the exception of DAB1 in TC-11, the rest of the genes in proximity of the breakpoints were not affected by the translocation however, the possible positional effect on their function was not evaluated.

TABLE 6
Genes within 100 kb of breakpoint sites assessed for TC-1 to TC-6

| TC-1 | TC-2 | TC-3 | TC-4 | TC-5 | TC-6 |
|---|---|---|---|---|---|
| AL118516.1 | AC007878.1 | AL031963.1 | AC118282.1 | AL359881.1 | AC083841.1 |
| TBC1D22A | AL731892.1 | AL031963.2 | AC118282.2 | AL359881.2 | AC083841.2 |
| TBC1D22A- | CYP4A11 | AL031963.3 | AC118282.3 | AL359881.3 | AC083841.3 |
| AS1 | CYP4B1 | AL133351.1 | AC118282.4 | CAMTA1 | AC083841.4 |
| | CYP4Z2P | AL133351.4 | MTCO3P39 | LINC01310 | AC090574.1 |
| | DYSF | AL133351.5 | MTND3P22 | PER3 | CYP11B1 |
| | RNU6-105P | BPHL | RPS14P7 | UTS2 | CYP11B2 |
| | ZNF638 | FAM136BP | SNX18P23 | VAMP3 | GML |
| | | LINC01011 | | Z98884.1 | LY6D |
| | | MIR7855 | | Z98884.2 | LYNX1 |
| | | NQO2 | | | LYPD2 |
| | | PLEKHG3 | | | PRDM14 |
| | | RIPK1 | | | RN7SL675P |
| | | RNA5SP201 | | | RNA5SP270 |
| | | SERPINB6 | | | RNU1-101P |
| | | SERPINB8P1 | | | SDCBPP2 |
| | | SPTB | | | SLURP2 |
| | | | | | SUMO2P20 |
| | | | | | ZNHIT1P1 |

Table 7 shows genes within 100 kb of breakpoint sites, as assessed for TC-7 to TC-11.

TABLE 7
Genes within 100 kb of breakpoint
sites assessed for TC-7 to TC-11

| TC-7 | TC-8 | TC-9 | TC-10 | TC-11 |
|---|---|---|---|---|
| AC007663.1 | CAND1 | AC090198.1 | AC110053.1 | DAB1 |
| AC007663.3 | GGTA2P | AC090922.1 | AC090503.2 | AL390243.1 |
| AC007663.4 | OR10D1P | AP006216.1 | | |
| AC007731.1 | OR10D3 | AP006216.2 | | |
| AC007731.3 | OR10N1P | APOA1 | | |
| AC007731.5 | OR8D1 | APOA1-AS | | |
| AP006216.1 | OR8F1P | APOA4 | | |
| AP006216.2 | OR8G1 | APOA5 | | |
| APOA1 | OR8G2P | APOC3 | | |
| APOA1-AS | OR8G3P | BUD13 | | |
| APOA4 | OR8G5 | MIR6844 | | |
| APOA5 | OR8G7P | MTSS1 | | |
| APOC3 | RAB11AP2 | NDUFB9 | | |
| BUD13 | SLC5A4P1 | RNF139 | | |
| DGCR6L | VWA5A | RNF139-AS1 | | |
| MIR1286 | | SIK3 | | |
| RNU6-225P | | TATDN1 | | |
| RTN4R | | TRMT12 | | |
| SCARF2 | | ZPR1 | | |
| SIK3 | | | | |
| USP41 | | | | |
| ZNF74 | | | | |
| ZPR1 | | | | |

Table 1 provides results of comprehensive whole genome copy number analysis of the embryos and carrier status for the rearrangement, using nanopore sequencing-breakpoint specific cBP-PCR. 55/82 embryos were diagnosed as aneuploid/unbalanced and 27/82 as euploid/balanced. 11 of the 27 euploid embryos were identified as balanced translocation carriers. No embryos were found to show mosaicism in this cohort of embryos. Nine euploid, non-carrier embryos have been transferred to date, of which 5 resulted in healthy live births, 2 in an ongoing pregnancy, 1 in a miscarriage and 1 in no implantation. cBP-PCR, Sanger sequencing and cytogenetic testing on five babies born to date confirmed 100% concordance with preimplantation testing results (see FIG. 9 to FIG. 14).

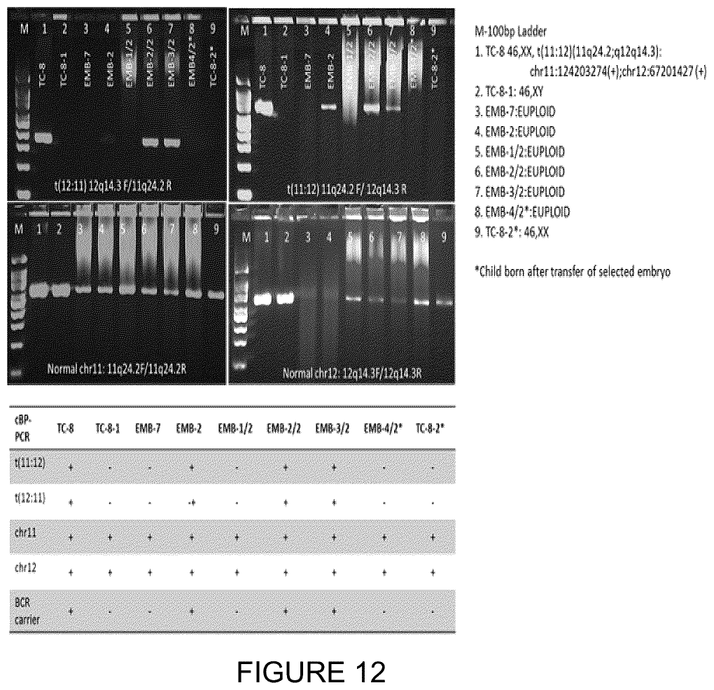

FIG. 11 to FIG. 14 show results from custom breakpoint (cBP) PCR in 3 couples undergoing PGT-SR. Comprehensive PGT-SR on the embryos was performed using the assay based on breakpoint mapping of parental samples using a MINION™ sequencer. FIG. 11 (TC-3) and FIG. 12 (TC-8) show preimplantation and postnatal results from cBP-PCR on children born after embryo transfer are in full concordance.

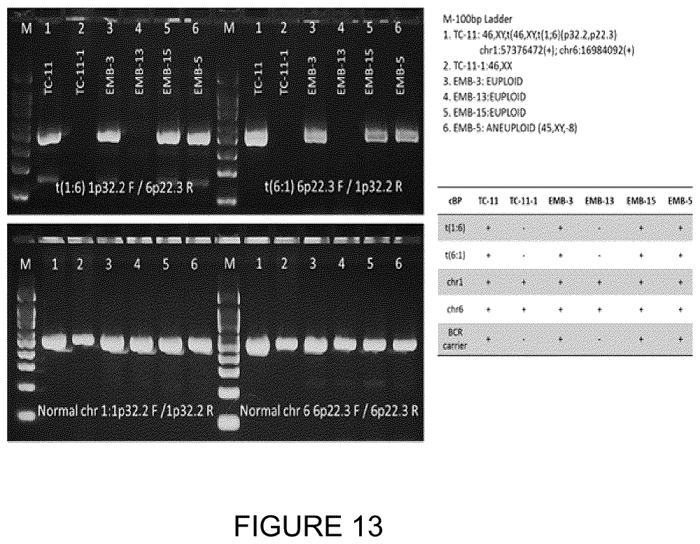

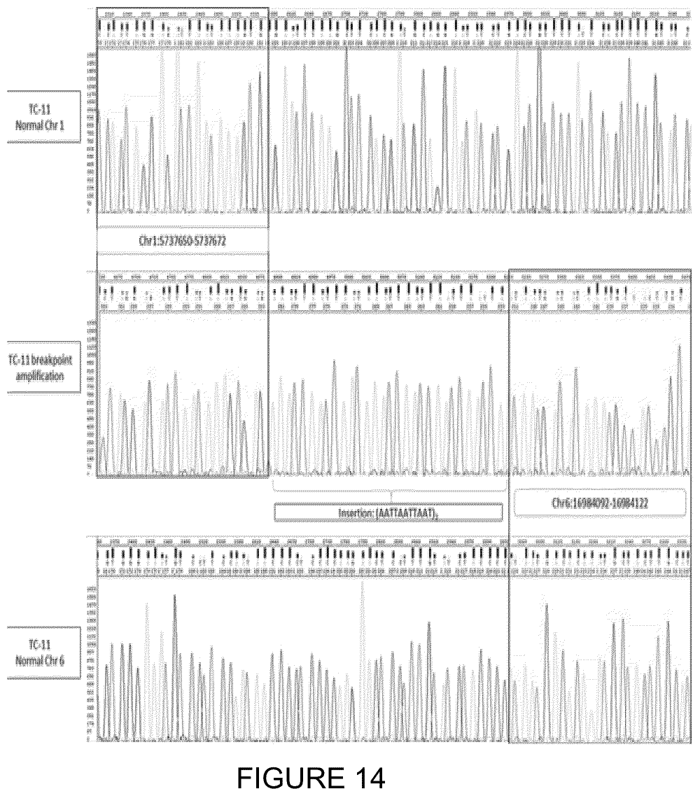

In FIG. 13 and FIG. 14, Sanger sequencing of the breakpoints of TC-11 revealed insertion of 22 bp at junction site, that was transmitted to both embryos balanced translocation carriers (EMB-3 and EMB-15). (EMB=embryo, *Postnatal results from a child born after embryo transfer selected with PGT-SR).

Standard Short Read PGT-SR Analysis is not Able to Distinguish BCR Carriers from Non-Carrier Embryos.

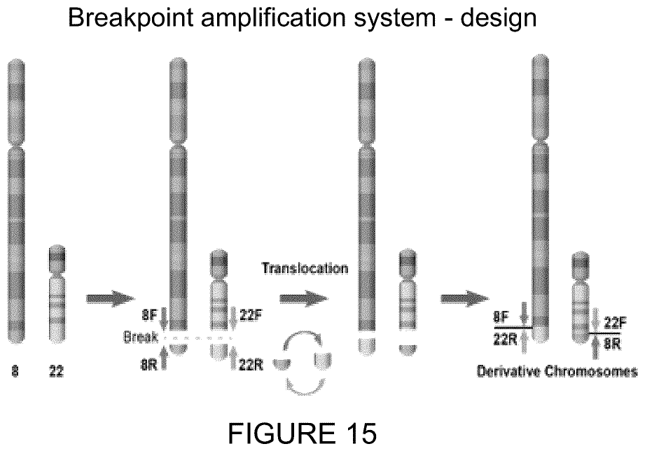

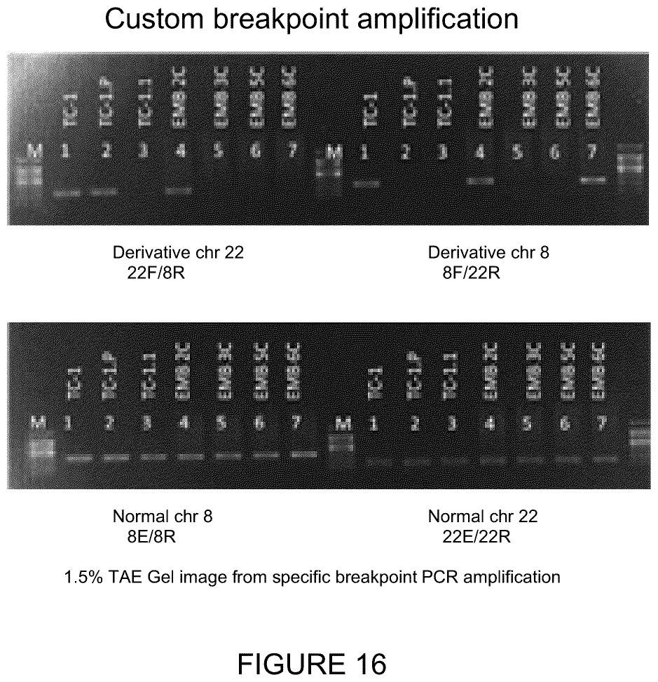

All embryos (n=82) from 11 couples with BCR were also tested using standard PGT-SR short read whole genome low pass sequencing to determine if they were abnormal (unbalanced or aneuploid), mosaic or euploid/balanced (Table 1). As discussed above, this approach is not able to distinguish balanced BCR carrier from non-carrier embryos. In addition, the resolution limit of this technique does not allow detection of unbalanced aberrations smaller than 10 Mb. For case TC-1 (46,XX,t(8:22)(q24.3,q13.33), with a history of 4 miscarriages and an affected child with Phelan-McDermid Syndrome (22q13 deletion, [arr[hg19]:8q24.3 (140,366,315-146,295,771)×3, 22q13.31q13.33 (47,254,703-51,176,099)×1p, short read sequencing failed to detect the Adjacent-1 unbalanced segregation. The application of a diagnostic strategy for comprehensive PGT-SR using nanopore sequencing is described in detail in FIG. 15 and FIG. 16. This strategy is applicable to the couple described as TC-1.

Standard NGS-based PGT-SR diagnosed 3 of the embryos to be balanced/euploid (EMB 2: 46,XX, 3: 46,XX and 5: 46,XX) and one aneuploid (EMB 6: 48,XY,+16,+21). Nanopore-based CNV analysis produced identical results and identified EMB 2 as euploid (46,XX) and EMB 6 as aneuploid (48,XY,+16,+21, del 8q24.3, dup22q13.31). cBP PCR determined that 1 of the 3 euploid embryos was a balanced carrier (EMB 2 46,XX, t(8,22)(q24.3,q13.31)), and the other 2 (EMB 3 and 5, 46,XX) were non-carriers. EMB 6 in addition to the trisomy 16 and 21 had inherited an unbalanced translocation resulting in deletion of chr8q24.3 and duplication of 22q13.31 (see FIG. 7 and FIG. 8).

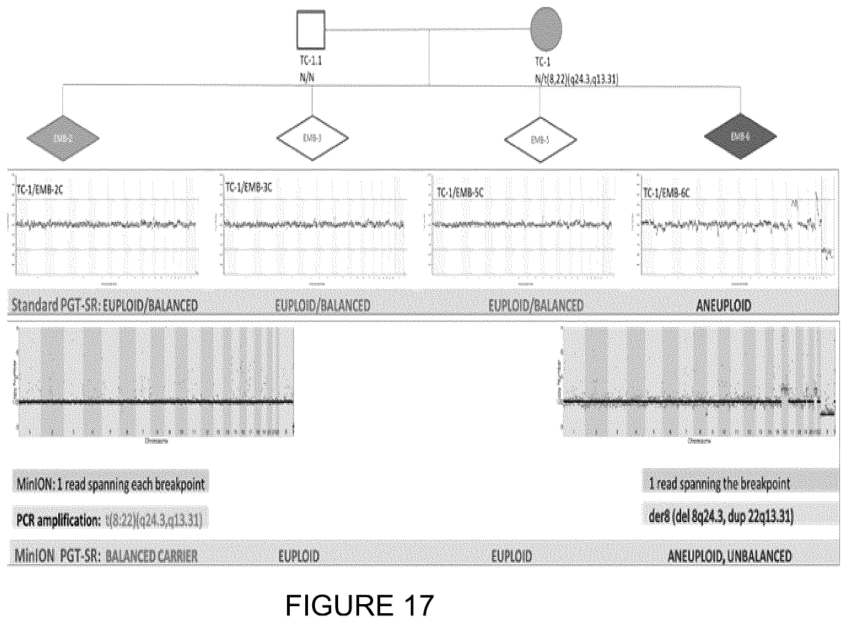

The Sanger sequencing single base resolution pertaining to data presented in FIG. 15 to FIG. 17 involved the following:
Normal chr 22, amplification primer set 22F/22R, chr22:46851962+46852103 (142 bp), Chr 22 breakpoint 46,852,062;
Normal chr 8, amplification primer set 8F/8R, chr8:139353389+139353718 (330 bp), Chr 8 breakpoint 139,353,588; and
Derivative t(22:8) amplification primer set 22F/8R, chr22:46851962+46852062: chr8:139353588+139353718 (t(22:8)(46852,062:139,353,588).

SNP array haplotyping and nanopore-based balanced translocation carrier discrimination showed full concordance. SNP array haplotyping was performed using a 5 Mb region around the breakpoint as described previously (Treff et al., 2016; Zhang et al., 2017). Breakpoint location for each BCR determined by nanopore sequencing was utilized to define the region needed to derive the haplotype associated with each translocation. DNA from the affected child of TC-1 and all unbalanced embryos from the remaining patients were used as references to map the rearranged chromosomes. The results regarding embryo carrier status that were determined by nanopore sequencing and cBP-PCR were fully concordant with haplotype segregation analysis of SNP arrays.

Gene Disruption by Translocation.

Breakpoints associated with balanced translocation of patient TC-11 t(1:6), disrupted intron 1 of DAB1 gene (Chr 1: 56,994,778-58,546,734 reverse strand, GRCh38:CM000663.2, OMIM #615945: Spinocerebellar ataxia 37, autosomal dominant inheritance). The gene is part of the reelin (RELN; OMIM 600514) signaling pathway that plays a critical role in the correct positioning of neurons within the developing brain. DAB1 serves as an intracellular adaptor that is tyrosine phosphorylated when reelin binds to the lipoprotein receptors VLDLR and APOER2 (LRP8) on the surface of neurons as shown in animal studies (Huang et al., 2005). DAB1 has complex expression because it contains several alternative first exons that can result in transcripts with variable 5-prime UTRs (Seixas et al., 2017). Various transcripts were found in multiple human central nervous system regions, including the cerebellum, and all showed higher expression in human fetal brain tissue compared to adult brain. DAB1 gene spans more than 1.2 Mb where exons 2 through 14 span more than 294 kb and contain the coding region for the major DAB1 isoform (Bar et al., 2003). Exon 15 contains the 3-prime UTR. Multiple 5-prime exons are spread over a 961-kb region and produce 6 alternative 5-prime UTRs. The most complex 5-prime UTR, UTR 1B, is made up of 7 exons.

The breakpoint on chr 1 of TC11 disrupts the gene however because of the complex expression and usually not translated exon 1 this patient has no phenotype associated with spinocerebellar ataxia.

Conclusion

These data illustrate that the comprehensive method described herein can discriminate between euploid BCR carrier and non-carrier embryos by accurate delineation of the chromosomal rearrangement in parent BCR carriers. A preimplantation test to determine translocation carriers can be used to the benefit of those pursuing reproductive technologies.

### Example 2

Targeted Sequencing of Breakpoint Regions in Cases with Reciprocal Translocations, Deletions, Duplications, Peri- and Para-Centric Inversions

High resolution mapping of the breakpoint regions of reciprocal translocations, deletions, duplications, perk and para-centric inversions allows for resolution of the exact location of the breakpoints, elucidating the effect of the chromosomal rearrangements on the chromosomal architecture, as well as on the genes and regulatory elements in the adjacent regions. Using long-read nanopore sequencing targeting the regions of interest, we were able to resolve breakpoints of balanced reciprocal translocations and complex deletions of the alpha thalassemia gene and the CYP21A2 gene. This targeted approach is applicable to detect the breakpoints in embryos without the need to construct breakpoint PCR “bpPCR”. Targeted sequencing is a directed sequence-based selection of reads that will be preferentially sequenced on a GRIDION™ sequencer, MINION™ sequencer, or FLONGLE™ (Oxford Nanopore Technologies, Oxford UK), sequencing adaptor using nanopore sequencing. FLONGLE™ sequencing. The library preparation comprises PCR free ligation using a LSK 114 kit (Oxford Nanopore Technologies, Oxford UK) and sequencing on R10 flow cell.

The regions of interest are defined to include the cytogenetic band and flanking 5 Mb upstream and downstream for a total of 25-45 Mb per region. The regions are defined in a “.bed” file that the sequencer uses to preferentially sequence the DNA strands that align to these regions. For reciprocal translocations the .bed file needs to have at least 2 regions that comprise of the cytobands with the breakpoints from the karyotype result. For all other deletions or duplications that do not involve other chromosomes the .bed file should include only the region with the mutation and flanking regions. This approach provides 4-10× more reads targeting the region of interest, allowing for higher fidelity of breakpoint reads, direct application to embryos, and use of this approach for variable sizes of deletions and duplications including de novo variant detection. Adjacent regions added to sequencing allows for haplotype generation and added information in resolving the carrier status in embryos.

3 μg of hmwDNA was sheared to ˜10-20 kb fragments using G-tubes (Covaris). Genomic libraries were prepared using the Oxford Nanopore 1D-ligation library prep kit SQK-LSK114 following ONT protocol (Oxford Nanopore Technologies, UK). A 0.4× (instead of 1×) AMPure magnetic beads (Beckman Coulter, A63881) clean-up was introduced after the end-repair steps (NEBNext Ultra II end-repair/dA-tailing module (New England BioLabs; E7546)) in the protocol to remove small DNA fragments. For 1D library the ligation reaction was prepared as follows: 30 μL of water (DNA), 20 μL of adaptor mix and 50 μL of blunt/TA ligase master mix (New England BioLabs; M0367).

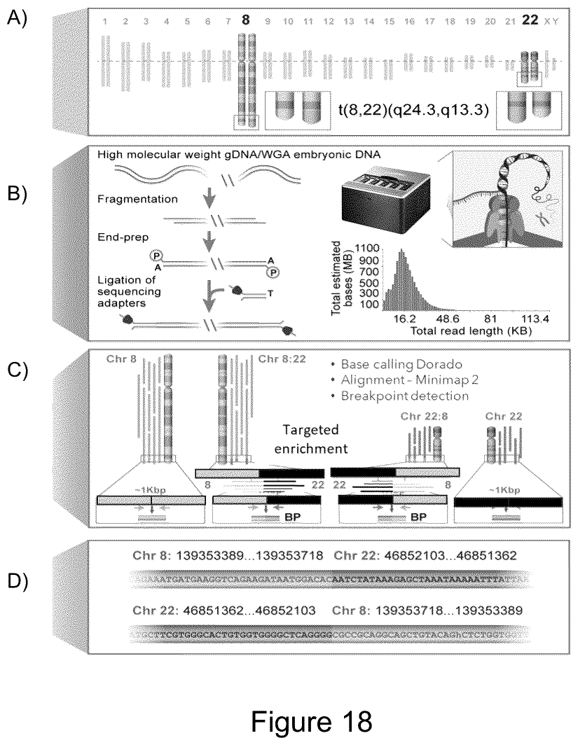

Ligation was carried out at room temperature for 75 min (instead of 15 min). The AMPURE™ XP beads (Beckman Coulter) were re-suspended at room temperature by vortexing. 40 μL of beads were added into the ligation product. The beads were washed twice with 140 μL of adaptor bead binding buffer and eluted in 25 μL of elution buffer. The eluted product was the adaptor-ligated library that was prepared for sequencing using the pre-sequencing mix and loading beads used in nanopore sequencing. Each genomic library was loaded and sequenced on a R10 flowcell supported by MINKNOW™ software (ONT) for 72 hours on GRIDION™ sequencer using MINKNOW™ software (ONT). The mode of adaptive sequencing was used with reference enrichment and used of bed file with genomic coordinates to include the cytogenetic band and flanking 5 Mb upstream and downstream for a total of 25-45 Mb per region. The total technician hands-on time for library preparation was 30 min with 4 hours overall time (FIG. 18).

Nanopore Sequencing Data Analysis for Detection of Breakpoints.

An analytical pipeline was developed for breakpoint detection which involves base calling, alignment, data quality filtering, local search and extraction of reads aligning to candidate BCR regions determined by karyotyping in all patients, followed by visual inspection. Basecalls were generated from the GRIDION™ sequencer data using DORADO™. Reads passing the basecaller's quality filter (mean quality score ≥7) were aligned to the hg38t2t human reference with Minimap2, version 2.1-r311 (Li, 2018). Each sample's run-level alignments were merged into a single per sample alignment (BAM format) using samtools. A local search was done for supporting reads in the aligned GRIDION™ sequencer data followed by visual inspection with the IGV genome browser (ivg.org).

FIG. 18 illustrates a workflow for breakpoint detection in BCR carriers using parent karyotype (genetic report) guided approach. As an example, BCR regions from TC-1 8q 24.3 and 22811.2 were used to enrich the sequencing reads aligning to the specific regions using ONT adaptive sequencing. The enrichment of the regions is 4-10 times higher than the average sequencing depth allowing for direct detection of breakpoints. Panel A illustrates regions of interest, t(8,22)(q24.3,q13.3). Panel B illustrates the processing, as high molecular weight gDNA/WGA embryonic DNA undergoes fragmentation, end-prep, ligation of sequence adapters. Total estimated bases (Mb) having a maximum of about 1100, being plotted against total read length (kb) having an average of about 16. Panel C illustrates targeted enrichment (Base calling DORADO™; Alignment—Minimap 2; Breakpoint detection). Panel D illustrates Chr 8:22 and Chr 22:8 from the targeted enrichment.

This analysis pipeline allowed confirmation of breakpoints for abnormalities previously discovered through cytogenetics and sequencing with a long read MINION™ sequencer with a mean genome coverage of 6.5×, and an average read length of 14 kb (see Table 8).

Metrics considered for targeted enrichment for BCR reciprocal translocation detection are outlined in Table 8.

TABLE 8
Targeted Long-Read Nanopore Sequencing Metrics

| | | | | | | Target | Number of |
|---|---|---|---|---|---|---|---|
| | | Number | Number | Average | Raw | enrichment of | putative |
| | No of | of reads | of bases | read length | Coverage | breakpoint | breakpoint |
| Sample | Flowcells | (M) | (GB) | (Bp) | (X) | cytoband (x) | reads |
| TC-1 t(8;22) | 1 | 16.2 | 10.6 | 13952.12 | 6.21 | Chr8:32 | 10 |
| (q24.3;q11.2) | | | | | | Chr22:24 | |
| TC-11 t(1;6) | 1 | 14.5 | 16.43 | 14457.75 | 7.98 | Chr1:34 | 12 |
| (p32.2;p22.3) | | | | | | Chr6:36 | |
| TC-12 t(12, 17) | 1 | 15.5 | 10.9 | 15046.43 | 5.7 | Chr12:45 | 6 |
| (q24.33;q23.1) | | | | | | Chr17:23 | |
| PGTM-1 | 1 | 13.2 | 10.9 | 9845.41 | 5.3 | Chr16 | 5 |
| (HBA1/HBA2 | | | | | | [p13.3]:22 | |
| del)[aa/--) | | | | | | | |

The same workflow as illustrated in this example may also be applied for detection of rearrangements such as insertions, deletions, inversions, paracentric inversion, pericentric inversion, duplications, translocations, reciprocal translocations, Robertsonian translocations in genes or genomic regions with defined coordinates. Such rearrangements may be detected if occurring de novo.

### Examples Only

In the preceding description, for purposes of explanation, numerous details are set forth in order to provide a thorough understanding of the embodiments. However, it will be apparent to one skilled in the art that these specific details are not required.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

### REFERENCES

All publications, patents and patent applications mentioned in this specification are indicative of the level of skill those skilled in the art to which this invention pertains and are herein incorporated by reference to the same extent as if each individual publication, patent, or patent application was specifically and individually indicated to be incorporated by reference.
Aristidou C, Koufaris C, Theodosiou A, et al. (2017) Accurate breakpoint mapping in apparently balanced translocation families with discordant phenotypes using whole genome mate-pair sequencing. PLoS One 12(1), e0169935.
Bar I, Tissir F, Lambert de Rouvroit C, De Backer O, Goffinet A M (2003). The gene encoding disabled-1 (DAB1), the intracellular adaptor of the Reelin pathway, reveals unusual complexity in human and mouse. The Journal of biological chemistry 278, 5802-12.
Cretu Stancu M, van Roosmalen M J, Renkens I, et al. (2017) Map-ping and phasing of structural variation in patient genomes using nanopore sequencing. Nat Commun 8, 1326.
Darwish, E. & Magdi, Y. Artificial shrinkage of blastocoel using a laser pulse prior to vitrification improves clinical outcome. Journal of assisted reproduction and genetics 33, 467-471, doi:10.1007/s10815-016-0662-z (2016).
Garvin T, Aboukhalil R, Kendall J, et al. (2015) Interactive analysis and assessment of single-cell copy-number variations. Nature methods 12, 1058-60.
Halgren C, Nielsen N M, Nazaryan-Petersen L, et al. (2018) Risks and recommendations in prenatally detected de novo balanced chromosomal rearrangements from assessment of long-term outcomes. Am J Hum Genet 102, 1090-103.
Handyside A H, Harton G L, Mariani B, et al. (2010) Karyomapping: a universal method for genome wide analysis of genetic disease based on mapping crossovers between parental haplotypes. Journal of medical genetics 47, 651-8.
Huang Y, Shah V, Liu T, Keshvara L. (2005) Signaling through Disabled 1 requires phosphoinositide binding. Biochemical and biophysical research communications 331, 1460-8.
Li H. (2018) Minimap2: pairwise alignment for nucleotide sequences. Bioinformatics (Oxford, England) 34, 3094-100.
Li H, Handsaker B, Wysoker A, et al. (2009) The Sequence Alignment/Map format and SAMtools. Bioinformatics (Oxford, England) 25, 2078-9.
Madjunkova S et al. Non-invasive preimplantation genetic testing for monogenetic diseases and aneuploidies using cell free embryonic DNA, in: American Society of Human Genetics 68th Annual Meeting, San Diego, p 1270, Abstract 3007T (2018).
Munné S, Sandalinas M, Escudero T, Fung J, Gianaroli L, Cohen J. (2000) Outcome of preimplantation genetic diagnosis of translocations. Fertil Steril 73, 1209-18.
Seixas A I, Loureiro J R, Costa C, et al. (2017) A Pentanucleotide ATTTC Repeat Insertion in the Non-coding Region of DAB1, Mapping to SCA37, Causes Spinocerebellar Ataxia. American journal of human genetics 101, 87-103.
Tan Y Q, Tan K, Zhang S P, et al. (2013) Single-nucleotide polymorphism microarray-based preimplantation genetic diagnosis is likely to improve the clinical outcome for translocation carriers. Human reproduction (Oxford, England) 28, 2581-92.
Treff N R, Thompson K, Rafizadeh M, et al. (2016) SNP array-based analyses of unbalanced embryos as a reference to distinguish between balanced translocation carrier and normal blastocysts. Journal of assisted reproduction and genetics 33, 1115-9.
Untergasser A, Cutcutache I, Koressaar T, et al. (2012) Primer3—new capabilities and interfaces. Nucleic acids research 40, e115.
Vermeesch J R, Voet T, Devriendt K. (2016) Prenatal and pre-implantation genetic diagnosis. Nat Rev Genet 17, 643-56.
Wang L, Shen J, Cram D S, et al. (2017) Preferential selection and transfer of euploid noncarrier embryos in preimplantation genetic diagnosis cycles for reciprocal translocations. Fertility and sterility 108, 620-7.e4.
Wei S, Weiss Z R, Gaur P, Forman E, Williams Z. (2018) Rapid preimplantation genetic screening using a handheld, nanopore-based DNA sequencer. Fertility and sterility 110, 910-6.e2.
Zhang S, Lei C, Wu J, et al. (2017) The establishment and application of preimplantation genetic haplotyping in embryo diagnosis for reciprocal and Robertsonian translocation carriers. BMC medical genomics 10, 60.

1. A method of determining carrier status of an embryo for a chromosomal rearrangement prior to implantation of the embryo, said method comprising:
obtaining cells of the embryo from a trophectoderm biopsy at least day 4 post in vitro fertilization;
conducting long-read nanopore sequencing and data analysis of the DNA of the cells and the DNA of the parent carrier of chromosomal rearrangement to detect at least one breakpoint, wherein the sequencing is conducted on DNA fragments prepared from the DNA of the parent carrier of chromosomal rearrangement and from the DNA of the cells, wherein said DNA fragments comprise ultra long fragments of 50 kb or greater, and said DNA fragments comprise an average fragment length of from about 5 to 20 kb, wherein the chromosomal rearrangement of the parent carrier comprises a translocation, reciprocal translocation, deletion, insertion, duplication, pericentric inversion, or paracentric inversion;
preparing customized primers specific to the breakpoint;
employing the customized primers in a polymerase chain reaction customized to the breakpoint (cBP-PCR) to determine whether the breakpoint is indicative of chromosomal rearrangement;
determining on the basis of cBP-PCR whether the embryo status is chromosomal rearrangement carrier or chromosomal rearrangement noncarrier; and
determining, on the basis of Sanger sequencing, whether the chromosomal rearrangement carrier embryo is fully balanced.

2. The method of claim 1, wherein the cells from trophectoderm biopsy comprise 3 to 10 trophectoderm cells.

3. The method of claim 1, wherein the cells are obtained from the embryo at day 5 or 6 post in vitro fertilization.

4. The method of claim 1, wherein in the step of conducting long-read nanopore sequencing, the DNA fragments are prepared and sequenced having an average length of from about 8 to 15 kb.

5. The method of claim 1, wherein in the step of conducting long-read nanopore sequencing, the DNA fragments are prepared and sequenced comprising ultra long fragments of 100 kb or greater.

6. The method of claim 1, wherein the long-read nanopore sequencing is conducted using a real-time long-read nucleic acid sequencer for up to 48 hours.

7. The method of claim 6, wherein a chromosomal copy-number variation (CNV) plot is generated from the long-read nanopore sequencing.

8. The method of claim 1, wherein multiple breakpoints are detected, and wherein customized primers are prepared for each breakpoint.

9. The method of claim 1, wherein the Sanger sequencing is conducted to determine full balance at single base resolution.

10. The method of claim 1, wherein the breakpoint is detected in a highly repetitive genomic region.

11. The method of claim 1, further comprising implantation of the embryo into a human subject if chromosomal rearrangement noncarrier status is indicated.

12. The method of claim 1, further comprising the step of freezing the embryo if chromosomal rearrangement noncarrier status is indicated.

13. The method of claim 12, further comprising the step of thawing the frozen embryo and implanting said embryo into a human subject.

14. The method of claim 1, wherein the chromosomal rearrangement carrier status of the embryo to be determined is a balanced chromosomal rearrangement, a cryptic imbalance or a complex rearrangement.

15. The method of claim 1, wherein the chromosomal rearrangement comprises a balanced chromosomal rearrangement selected from the group consisting of inversion and translocation.

16. A method of determining carrier status of an embryo for a chromosomal rearrangement prior to implantation of the embryo, said method comprising:
obtaining cells of the embryo from a trophectoderm biopsy at least day 4 post in vitro fertilization;
conducting long-read nanopore sequencing and data analysis of the DNA of the cells and the DNA of the parent carrier of the chromosomal rearrangement to detect at least one breakpoint;
preparing customized primers specific to the breakpoint;
employing the customized primers in a polymerase chain reaction customized to the breakpoint (cBP-PCR) to determine whether the breakpoint is indicative of chromosomal rearrangement;
determining on the basis of cBP-PCR whether the embryo status is chromosomal rearrangement carrier or chromosomal rearrangement noncarrier; and
determining, on the basis of Sanger sequencing, whether the chromosomal rearrangement carrier embryo is fully balanced.

17. The method of claim 16, wherein the cells from trophectoderm biopsy comprise 3 to 10 trophectoderm cells.

18. The method of claim 16, wherein the chromosomal rearrangement of the at least one carrier parent comprises a reciprocal translocation, a pericentric inversion, or paracentric inversion.

19. The method of claim 16, wherein in the step of conducting long-read nanopore sequencing, the DNA fragments are prepared and sequenced having an average length of from about 5 to 20 kb, from about 8 to 15 kb, or from about 8 to 10 kb.

20. The method of claim 16, wherein in the step of conducting long-read nanopore sequencing, the DNA fragments are prepared and sequenced comprising ultra long fragments of 50 kb or greater, or 100 kb or greater.
